(19) United States
(12) Patent Application Publication
(10) Pub. No.: US 2025/0186143 A1
(43) Pub. Date: Jun. 12, 2025

(54) ROBOTIC SURGERY SYSTEMS, DEVICES, AND METHODS OF USE

(71) Applicant: Levita Magnetics International Corp., Mountain View, CA (US)

(72) Inventors: Greg LIU, Cupertino, CA (US); Joseph DENNIS, Millbrae, CA (US); Alberto RODRIGUEZ-NAVARRO, San Francisco, CA (US); Vicente OPASO-VALENZUELA, Santiago (CL); Oscar CERDA, Mountain View, CA (US); Laurence GOLBORNE, Mountain View, CA (US); Chauncey GRAETZEL, Palo Alto, CA (US)

(21) Appl. No.: 18/892,366

(22) Filed: Sep. 21, 2024

Related U.S. Application Data
(60) Provisional application No. 63/540,007, filed on Sep. 22, 2023.

(51) Int. Cl. A61B 34/30; A61B 17/00; A61B 17/02; A61B 17/068; A61B 17/08

(52) U.S. Cl. CPC A61B 34/30; A61B 17/02; A61B 17/068; A61B 17/083; A61B 2017/00477; A61B 2017/00973; A61B 2034/303; A61B 2034/305

(57) ABSTRACT

Described here are systems, devices, and methods useful for minimally invasive surgical procedures performed by a single operator. A robotic surgery system may include a robotic arm having a coupling mechanism for releasably coupling to an end effector connector. Methods for removably coupling the robotic surgery system may include coupling the end effector connector to the coupling mechanism, actuating the coupling mechanism to release the end effector connector, and receiving the end effector connector. The coupling, actuating, and receiving may be performed with a single hand of an operator. Additionally, devices for performing minimally invasive surgery may include a robotic arm input device having independently controllable foot-actuated switches. Methods for using the input device may include receiving a robotic arm control signal based on single-footed operation of the input device and controlling a movement of the robotic arm accordingly.

### TECHNICAL FIELD

This application claims priority to U.S. Provisional Patent Application Ser. No. 63/540,007 filed Sep. 22, 2023, the contents of which is incorporated herein by reference in their entirety for all purposes.

### TECHNICAL FIELD

Devices, systems, and methods herein relate to minimally invasive procedures using a robotic surgery system that may be operated by a single hand or single foot of an operator.

### BACKGROUND

Many surgical procedures utilize or incorporate minimally invasive approaches to minimize the number and size of incisions that are made in a patient. Minimally invasive procedures such as endoscopic, laparoscopic, and thoracoscopic procedures may be associated with lower pain, quicker post-surgical recovery, shortened hospitalization, and reduced complications when compared to open surgical procedures. Traditional minimally invasive robotic surgery procedures are generally performed by two skilled surgeons (e.g., operators). During these procedures, a primary surgeon may perform the surgical tasks (e.g., dissection, clipping, cutting, stapling, etc.) and a secondary surgeon assists in these functions. The primary surgeon may be located at a console outside of a sterile field while the secondary surgeon may be located within the sterile field to assist by, for example, changing the instruments (e.g., end effectors) coupled to a robotic surgery system. The secondary surgeon may also assist the primary surgeon by holding an instrument in each hand, such as an optical sensor (e.g., camera) in a first hand and a retractor in a second hand. Accordingly, it may be desirable to provide a robotic surgery system that may be less cumbersome and resource intensive than those currently in use.

### SUMMARY

Described herein are systems, devices, and methods useful for minimally invasive surgical procedures. In some variations, the procedures described herein may be performed by a single operator absent additional assistance from another operator.

A robotic surgery system may include a coupling mechanism at a distal end of a robotic arm and an end effector connector. The coupling mechanism may include a cylindrical housing, an actuator disposed within the cylindrical housing, a sleeve disposed around the cylindrical housing, and one or more projections extendable from and retractable into a sidewall of the cylindrical housing. The end effector connector may include a distal end configured to receive an end effector and a proximal end including an annular portion configured to releasably couple to the cylindrical housing, and the annular portion may include one or more grooves configured to receive the one or more projections of the cylindrical housing. In some variations, the one or more grooves may be disposed along an inner circumference of the annular portion. In some variations, the sleeve may be configured to translate over the cylindrical housing to retract the one or more projections into the cylindrical housing in response to actuation of the actuator. The system may further include a sterile drape including a first side and a second side opposite the first side, and the sterile drape may be configured to be disposed between the coupling mechanism and the end effector connector such that the first side faces the coupling mechanism and the second side faces the end effector connector. The end effector connector may further include a handle coupled and extending between the distal end and the annular portion. Additionally, the end effector may include one or more of a visualization device, a grasper, a retractor, a magnetic positioning device, a sensor, an intracavity device, a delivery device, a retrieval device, a stapler, a clip applier, and an electrocautery hook.

The actuator may be coupled to the one or more projections and configured to retract the one or more projections into the sidewall of the cylindrical housing in response to actuation of the actuator. In some variations, the annular portion may be decoupled from the cylindrical housing when the one or more projections are retracted into the sidewall of the cylindrical housing.

The sleeve of the coupling mechanism may be configured to align the annular portion in a predetermined rotational configuration relative to the cylindrical housing. In some variations, a distal end of the sleeve may include one or more protrusions and indentations, and the annular portion may include one or more complementary indentations and protrusions.

The end effector connector may be configured to releasably couple the end effector to the robotic arm. In some variations, the robotic arm may be configured to move the end effector within a surgical site of a patient when the end effector is coupled to the end effector connector.

Another robotic surgery system may include a robotic arm, an end effector, an adapter for the end effector comprising a lumen configured to receive the length of the end effector therethrough, and a connector for the end effector. The connector may include a first end configured to releasably couple to the robotic arm and a second end with a clamp configured to receive a length of the end effector therein. The clamp may be configured to receive the end effector via the adapter to releasably couple the end effector to the connector. In some variations, the adapter may include a substantially cylindrical body that defines the lumen. In some variations, the end effector may include one or more of a visualization device, a grasper, a retractor, a magnetic positioning device, a sensor, an intracavity device, a delivery device, a retrieval device, a stapler, a clip applier, and an electrocautery hook. For example, a visualization device may include an endoscope.

In some variations, the end effector may have a cylindrical shape. In some variations, a diameter of the end effector may be about equal to or less than 10 mm. In some variations, the lumen of the adapter may have a diameter that is about equal to or greater than the diameter of the end effector.

In some variations, the clamp may include a base and a moveable portion that is coupled to and moveable relative to the base. The base may have a first recess configured to receive a first portion of the adapter, and the moveable portion may have a second recess configured to receive a second portion of the adapter. The moveable portion may be rotatable relative to the base. The clamp may be configured to transition from open configuration and a closed configuration to secure the end effector therein via the adapter. In some variations, clamp may further include a lock configured to maintain the closed configuration of the clamp. The lock may be rotatably coupled to one or both of the base and the moveable portion. In some variations, the lock may have an elongate lock body. A distal end of the elongate lock body may be configured to releasably engage a recess within the base of the clamp to maintain the clamp in the closed configuration.

Also described herein is a method for removably coupling a robotic surgery system. The method may first include coupling an end effector connector to a coupling mechanism, the coupling mechanism coupled to a distal end of a robotic arm, where the coupling mechanism may include a cylindrical housing defining a longitudinal axis, a sleeve disposed around the cylindrical housing, an actuator disposed within the cylindrical housing, and one or more projections extendable from and retractable into a sidewall of the cylindrical housing. The end effector connector may include an annular portion releasably coupled to the cylindrical housing, and the annular portion may include one or more grooves configured to receive the one or more projections of the cylindrical housing. Next, the method may include actuating the actuator with a first hand of an operator to retract the one or more projections within the sidewall of the cylindrical housing to release the annular portion from the cylindrical housing. Finally, the method may include receiving the annular portion with the first hand of the operator. Each of the coupling, actuating, and receiving may be performed by a single operator. In some variations, coupling the end effector connector to the coupling mechanism may include pressing the annular portion onto and about a circumference of the cylindrical housing. In some variations, actuating the actuator may include applying a force to a distal portion of the coupling mechanism with the first hand of the operator. Further, the method may include further disposing a sterile drape between the coupling mechanism and the end effector connector such that a first side of the sterile drape faces the coupling mechanism and a second, opposite of the sterile drape faces the end effector connector.

In some variations, the end effector connector may further include a handle coupled to the annular portion and an end effector housing coupled to the handle, and the end effector housing may be configured to releasably couple to an end effector. The end effector may include one or more of a visualization device, a grasper, a retractor, a magnetic positioning device, a sensor, an intracavity device, a delivery device, a retrieval device, a stapler, a clip applier, and an electrocautery hook. In some variations, the method may further include, prior to actuating the actuator, decoupling the end effector from the handle.

In some variations, the coupling may include aligning the annular portion in a rotational configuration relative to the sleeve of the coupling mechanism. The aligning may include aligning one or more protrusions and indentations of the annular portion relative to one or more complementary indentations and protrusions of the sleeve.

Also described herein is a support arm input device, the device generally including: a base having a proximal end, a distal end, a lateral portion, and a midfoot portion between the proximal end and the distal end, where the midfoot portion may be configured to receive a midfoot of an operator; a first forefoot actuator coupled to the distal end of the base, where the first forefoot actuator may be configured to generate a first support arm control signal corresponding to one or more of pitch, yaw, and roll of a support arm; a second forefoot actuator coupled to the lateral portion of the base, where the second forefoot actuator may be configured to generate a second support arm control signal corresponding to distal translation of the support arm relative to the operator; and a hindfoot actuator coupled to the proximal end of the base, where the hindfoot actuator may be configured to generate a third support arm control signal corresponding to proximal translation of the support arm relative to the operator. Each of the first forefoot actuator, the second forefoot actuator, and the hindfoot forefoot actuator may be configured to be independently actuated.

The first forefoot actuator may be configured to control one or more of pitch, yaw, and roll movement of an end effector of the support arm. In some variations, the first forefoot actuator may include a first forefoot housing releasably coupled to a first forefoot receptacle configured to receive the forefoot of the operator, and the first forefoot housing may include a plurality of forefoot switches. In some variations, each switch of the plurality of forefoot switches may be configured to be actuated via manipulation of the first forefoot receptacle by the forefoot of the operator.

The second forefoot actuator may include a second forefoot switch configured to be actuated by a forefoot of an operator. In some variations, the second forefoot actuator may include a second forefoot housing configured to receive the forefoot of the operator. Additionally, in some variations, second forefoot actuator may be configured to control distal translation of an end effector of the support arm relative to the operator. Further, the second forefoot actuator may be coupled to a distal end of the lateral portion.

The hindfoot actuator may include a hindfoot switch configured to be actuated by a hindfoot of the operator. In some variations, the hindfoot switch may include an adjustment mechanism configured to adjust a position the hindfoot switch along a longitudinal axis of the base. In some variations, the hindfoot actuator may include a hindfoot housing configured to receive a hindfoot of the operator. Moreover, the hindfoot actuator may be configured to control proximal translation of an end effector of the support arm relative to the operator.

In some variations, the support arm may be a first support arm and the device may be configured to control movement of the first support arm and a second support arm. The device may further include a third forefoot actuator coupled to a first forefoot housing, where the third forefoot actuator may be configured to generate a fourth support arm control signal for transferring transmission of the first, second, and third support arm control signals from the first support arm to the second support arm. In some variations, the third forefoot actuator may include a third forefoot switch configured to be actuated by an underfoot of the operator. In some variations, the third forefoot actuator may be coupled to an exterior surface of a first forefoot housing of the first forefoot actuator.

Further, the support arm input device may be in a robotic surgery system, where the system may also include the support arm and an end effector releasably couplable to the support arm. The end effector may include one or more of a visualization device, a grasper, a retractor, a magnetic positioning device, a sensor, an intracavity device, a delivery device, a retrieval device, a stapler, a clip applier, and an electrocautery hook. The system may additionally include an end effector connector configured to releasably couple the end effector to the support arm. Moreover, the support arm control signal may be configured to control movement of one or both of the support arm and the end effector.

Also described herein is a method for using a support arm input device, where the support arm input device may include a base having a proximal end, a distal end, a lateral portion, and a midfoot portion between the proximal end and the distal end, and where the midfoot portion may be configured to receive a midfoot of an operator. The method may include independently receiving one or more of: a first support arm control signal via actuation of a first forefoot actuator coupled to the distal end of the base, a second support arm control signal via actuation of a second forefoot actuator coupled to the lateral portion of the base, and a third support arm control signal via actuation of a hindfoot actuator coupled to the proximal end of the base. Next, the method may include controlling a movement of a support arm relative to a surgical space based on one or more of the first, second, and third support arm control signals. The first support arm signal may control one or more of pitch, yaw, and roll of the support arm. The second support arm signal may control distal translation of the support arm. The third support arm signal may control proximal translation of the support arm.

Also described herein is a method for determining a parameter for robotic surgery, including: defining a first reference point between an end effector and a support arm coupled thereto, positioning the end effector relative to a surface of a body within a surgical space, acquiring data on a position of the end effector relative to the surface of the body, and determining a second reference point between the end effector and the surface of the body based on the acquired data and the first reference point. In some variations, the body may be a body of a patient. Further, the second reference point may be an incision on the body of the patient. In some variations, acquiring the data may include actuating a control button to model the position of the end effector within the surgical space. The control button may be on, for example, the support arm. In some variations, a processor may be configured to model the position of the end effector. In some variations, the method may further include determining a dimension of the end effector based on the second reference point. The dimension may be a length of the end effector. Further, the determining may include calculating a distance between the first and second reference points. In some variations, the end effector may include a visualization device.

Another method for determining a parameter for robotic surgery may include: defining a first reference point between an end effector and a support arm coupled thereto. receiving a length of the end effector via a user interface, positioning the end effector relative to a surface of a body within a surgical space, and determining a second reference point between the end effector and the surface of the body based on the first reference point and the length of the end effector. In some variations, body may be a body of a patient. Additionally, the second reference point may be an incision on the body of the patient. In some variations, body may be an object. The object may include a planar surface. In some variations, the object may be a surgical table. In some variations, the body may include a marker. In some variations, the first reference point may be an intersection between the support arm and the proximal portion of the end effector. In some variations, the length of the end effector may be about 20 cm to about 60 cm.

Also described herein is a method for measuring an end effector, including defining a first reference point of an end effector, moving the end effector to a first position relative to a body to form a first angle between a longitudinal axis of the end effector and an axis of the body, moving the end effector to a second position relative to the body to form a second, different angle between the longitudinal axis of the end effector and the axis of the body, and determining a length of the end effector based on the reference point, the first angle, and the second angle. In some variations, the end effector may have an elongate body. The length of the end effector may be about 20 cm to about 60 cm. Further, the end effector may include one or more of a visualization device, a grasper, a retractor, a magnetic positioning device, a sensor, an intracavity device, a delivery device, a retrieval device, a stapler, a clip applier, and an electrocautery hook. In some variations, the visualization device may include an endoscope.

In some variations, moving the end effector to the first position and the second position may include intersecting a surface of the body with a distal tip of the end effector. The body may be a body of a patient, a marker, or an object, such as a surgical table. In some variations, the object may include a planar surface. Moving the end effector to the first position and the second position may include contacting the marker or the planar surface of the object with a distal tip of the end effector. The method may further include determining a third position of the end effector within the body of the patient during a surgical procedure based on the length of the end effector.

In some variations, the method may further include releasably coupling the end effector to a support arm configured to control movement of the end effector. A proximal portion of the end effector may be releasably coupled to the support arm. In some variations, the first reference point may include an intersection between the support arm and the proximal portion of the end effector. In some variations, the support arm may move the end effector between the first position and the second position. The support arm may be configured to be one or both of manually and mechanically actuated.

In some variations, determining the length of the end effector may include determining a second reference point between a distal tip of the end effector and a surface of the body and calculating a distance between the second reference point and the first reference point. The distance may be determined by triangulating first and second vectors defined by the first and second angles. In some variations, the body may be a body of the patient, and the second reference point may be an incision on the body of the patient.

Another method for measuring an end effector may include: forming an incision on a body of a patient, wherein the patient is within a surgical space, defining a first reference point between an end effector and a support arm coupled thereto within the surgical space, positioning the end effector relative to the incision such that a longitudinal axis of the end effector forms a first angle relative to an axis bisecting the incision, defining a first vector based on the first angle, adjusting a position of the end effector relative to the incision such that the longitudinal axis of the end effector forms a second, different angle relative to the axis bisecting the incision, defining a second vector based on the second angle, determining a second reference point between the end effector and the incision, where the second reference point may define a position of the incision within the surgical space, and determining a length of the end effector by calculating a distance between the first and second reference points.

Also described herein is a method for controlling movement of an end effector, including providing, via a display coupled to a controller, an image of a field of view of an operator, where the image may include an end effector and a reference marker overlaid onto or adjacent to the end effector, measuring a gaze of the operator using the controller, and actuating a support arm to move the end effector from a first position to a second position based on the measured gaze of the operator. In some variations, the reference marker may include one or more of a symbol, image, or text. When a patient is within the field of view, actuating the support arm to move the end effector from the first position to the second position within the image may include moving the end effector from a first position to a second position with respect to a body of the patient. Moreover, the end effector may include one or more of a visualization device, a grasper, a retractor, a magnetic positioning device, a sensor, an intracavity device, a delivery device, a retrieval device, a stapler, a clip applier, and an electrocautery hook.

In some variations, providing the image may include tracking the end effector and overlaying the reference marker onto or adjacent to the end effector in real-time. When a patient is within the field of view, tracking the end effector may include determining a location of the end effector when the end effector is both inside of and external to a body of the patient. In some variations, end effector may include an RFID tag that enables tracking of the end effector.

In some variations, actuating the support arm may include generating one of a plurality of support arm control signals in response to the gaze of the operator. The method may further include providing, within the image, a plurality of virtual actuators each configured to respond to the gaze of the operator to generate one of the plurality of support arm control signals. The plurality of virtual actuators may include a first virtual actuator configured to translate the support arm along a first axis within the image and a second virtual actuator configured to translate the support arm along a second, different axis within the image. In some variations, the first virtual actuator may be configured to enable translation of the support arm such that the end effector moves in a first direction along the first axis, the second virtual actuator may be configured to enable translation of the support arm such that the end effector moves in the first direction along the second axis, and the plurality of virtual actuators may further include a third virtual actuator configured to enable translation of the support arm such that the end effector moves in a second, opposite direction along the first axis, and a fourth virtual actuator configured to enable translation of the support arm such that the end effector moves in the second direction along the second axis. In some variations, the plurality of virtual actuators may further include at least one fifth virtual actuator configured to enable rotation of the support arm in one or more of pitch, roll, and yaw. Moreover, actuating the support arm may include directing the gaze of the operator at one of the plurality of virtual actuators for a time period. In some variations, the time period may be about 1 second to about 10 seconds.

In some variations, the controller may be configured to be worn over the eye of the operator. The controller may include one or more of a headset, goggles, glasses, and a contact lens.

In some variations, the controller may include a first controller, and the method may further include actuating an actuator on a second controller to activate the first controller. The actuator on the second controller may be configured to receive input from a foot of the operator to activate the first controller. In some variations, the second controller may include a foot pedal. In some variations, prior to actuating the actuator on the second controller, the method may include communicably coupling the first and second controller via a remote network.

### BRIEF DESCRIPTION OF THE DRAWINGS

FIG. 1 is a block diagram of an illustrative variation of a robotic surgery system.

FIGS. 2A-2D depict perspective views of an illustrative variation of a robotic surgery system. FIG. 2E-2H depict perspective views of an illustrative variation of an end effector housing release mechanism of a robotic surgery system. FIG. 2I depicts a top view of an illustrative variation of an end effector housing of a robotic surgery system.

FIGS. 3A-3D depict perspective views of an illustrative variation of a robotic surgery system. 3E depicts a perspective view of an illustrative variation of an end effector connector of a robotic surgery system. FIGS. 3F and 3G depict side views of an illustrative variation of a coupling mechanism of a robotic surgery system. FIG. 3H depicts a perspective view of an illustrative variation of a robotic surgery system.

FIGS. 4A-4C depict cross-sectional views of an illustrative variation of a robotic surgery system.

FIGS. 5A-5D are photographic representations of a variation of a robotic surgery system.

FIG. 6A depicts a perspective view of an illustrative variation of an input device of a robotic surgery system. FIGS. 6B-6D depict top views of an illustrative variation of an input device of a robotic surgery system. FIG. 6E depicts a front view of an illustrative variation of an input device of a robotic surgery system. FIG. 6F depicts a section view of an illustrative variation of an input device of a robotic surgery system FIG. 6G depicts a back view of an illustrative variation of an input device of a robotic surgery system.

FIG. 7 is a flowchart representation of an illustrative variation of a method for removably coupling a robotic surgery system.

FIG. 8 is a flowchart representation of an illustrative variation of a method for using a robotic arm input device.

FIG. 9A depicts a perspective view of an illustrative variation of an end effector connector of a robotic surgery system. FIG. 9B depicts a perspective view of an illustrative variation of a clamp of the end effector connector.

FIG. 10A depicts a perspective view of an illustrative variation of a clamp of an end effector connector in a closed configuration. FIG. 10B depicts a perspective view of an illustrative variation of the clamp of FIG. 10A in an open configuration.

FIG. 11 depicts a perspective view of an illustrative variation of another clamp of an end effector connector in a closed configuration.

FIG. 12 depicts a perspective view of an illustrative variation of yet another clamp of an end effector connector in a closed configuration.

FIGS. 13A and 13B depict perspective views of an illustrative variation of an end effector connector of a robotic surgery system.

FIG. 14 depicts a cross-sectional view of an illustrative variation of an adapter for use with an end effector connector.

FIGS. 15A and 15B depict a perspective views of an illustrative variation of an adapter within an end effector connector of a robotic surgery system.

FIG. 16 depicts a schematic diagram of an illustrative variation of a control system of a robotic surgery system.

FIG. 17 depicts an exemplary augmented image provided during a robotic surgery procedure.

FIG. 18 depicts a flow diagram of an illustrative variation of a method for controlling movement of an end effector.

FIG. 19 depicts a flow diagram of an illustrative variation of another method for controlling movement of an end effector.

FIG. 20 depicts a flow diagram of an illustrative variation of a method for determining a reference point for robotic surgery.

FIG. 21 depicts a flow diagram of an illustrative variation of another method for determining a reference point for robotic surgery.

FIG. 22 depicts a flow diagram of an illustrative variation of a method for measuring an end effector.

FIGS. 23A and 23B depict an exemplary representation of the variables used to determine surgical parameters for performing robotic surgery.

### DETAILED DESCRIPTION

Described here are systems, devices, and methods for use in minimally invasive surgical procedures confirmed to be performed by a single operator absent additional assistance from another operator. For example, the systems, devices, and methods described herein may improve minimally invasive robotic surgery by: enabling single operator operation of a robotic surgery system without a second operator; reducing sterile field management using a magnetic robotic surgery system; facilitating rapid tool (e.g., end effector) exchanges; improving operator ergonomics of a robotic surgery system; enhancing single operator control of a robotic arm (also referred to herein as “support arm”); facilitate rapid and versatile end effector coupling to the support arm; facilitating rapid and accurate end effector measurement and registration; providing hands free control of a support arm; enhancing visualization (e.g., virtual reality, augmented reality, extended reality) of a robotic surgical procedure; and monitoring patient safety with respect to the robotic surgery system.

While conventional robotic surgery systems require a first operator to be assisted by a less skilled second operator (e.g., scrub nurse) to perform various functions during a minimally invasive surgical procedure (e.g., a laparoscopic procedure), the systems and methods disclosed herein may not require a second skilled operator to assist the single operator. For example, the systems, devices, and methods described herein may enable rapid end effector cleaning and port changes with just the single operator without affecting a sterile field, thereby alleviating the need for aseptic technique. Additionally, the systems, devices, and methods detailed below may allow for single-handed or single-footed actuation by the operator, further simplifying use of the robotic surgery systems described herein.

Generally, the systems, devices, and methods described herein facilitate rapid end effector exchanges with a robot arm that may improve the speed and/or efficiency of a robotic surgical procedure. In some variations, the system may include a coupling mechanism at a distal end of a robotic arm and an end effector connector. The coupling mechanism may include a cylindrical housing, an actuator disposed within the cylindrical housing, a sleeve disposed around the cylindrical housing, and one or more projections extendable from and retractable into a sidewall of the cylindrical housing. The end effector connector (also referred to herein as “connector”) may include a distal end configured to receive an end effector and a proximal end including an annular portion configured to releasably couple to the cylindrical housing, and the annular portion may include one or more grooves configured to receive the one or more projections of the cylindrical housing. In some variations, the end effector connector may include a distal end having a clamp that is configured to receive and hold an end effector therein.

In some variations, the end effector connector may be configured to releasably couple to end effectors of various geometries and dimensions using an adapter, thereby accommodating a wider range of end effectors and procedures. For example, visualization devices may have different lengths and diameters. In some variations, an adapter may be configured to couple to the end effector at an intersection between the end effector and the end effector connector to adjust a size of the end effector such that it may fit within the clamp. For example, the adapter may be configured to receive the end effector within a lumen thereof to increase a dimension (e.g., a width or diameter) of at least a portion of the end effector so that the end effector may be secured within the clamp. Accordingly, an end effector connector may be configured to accommodate different end effector configurations which may provide one or more of increased versatility, reduced procedure time, and reduced cost.

In some variations, the end effector connector (“connector”) may function as a handle held by a single hand of the operator. For example, the end effector connector may be held and moved (e.g., hand guided) by the operator to reposition the end effector as desired. Moreover, the end effector may be released from the robot via the end effector connector to facilitate rapid tool changes and/or cleaning. In some variations, the end effector connector may have a distal end comprising a clamp configured to maintain an end effector therein. The system may further include an adapter configured to fit within the clamp. The adapter may include a lumen configured to receive the end effector therethrough such that the end effector is secured within the clamp via the adapter. Thus, the adapter may contribute to the flexibility of the systems herein because it may facilitate coupling of an end effector to a connector when the geometry of the end effector and connector clamp (e.g., respective widths or diameters thereof) are mismatched.

Furthermore, the configuration and network of controllers herein enable single operator control of one or more robotic arms and end effectors by freeing the hands and visual attention of the operator to be elsewhere. For example, a controller may comprise an input device configured to receive operator input to control one or more elements of the robotic surgery systems herein. For example, the input device may be communicably coupled to one or more robotic arms and/or one or more end effectors to control, for example, movement of the robotic arm(s) and/or end effector(s). In some variations, the input device may be configured to receive input from one or more body parts of an operator (e.g., hand, arm, foot, leg, head, eyes, etc.). In some variations, the controller may be configured to be worn over an eye of an operator, or may be configured to be positioned beneath a foot of the operator. In either case, both hands of the operator may remain free. In some variations, the input device may include a foot-operated device (e.g., a foot controller, foot switch, pedal). Such an input device input device may include a base for receiving a foot of an operator and a plurality of foot-operated switches (e.g., actuators) coupled to the base, where each switch corresponds to movement of a support arm and/or end effector. For example, a first switch may be configured to control rotational movement of the support arm and/or end effector, a second switch may be configured to control proximal translational movement of the support arm and/or end effector, and a third switch may be configured to control distal translational movement of the support arm and/or end effector. Using such an input device may include independently receiving one or more of: a first support arm control signal via actuation of a first forefoot actuator coupled to the distal end of the base, a second support arm control signal via actuation of a second forefoot actuator coupled to the lateral portion of the base, and a third support arm control signal via actuation of a hindfoot actuator coupled to the proximal end of the base; and controlling a movement of a support arm based on one or more of the first, second, and third support arm control signals. In some variations, the systems herein may include one or more controllers or input devices, such as a first controller or input device (e.g., a foot-operated input device) and a second controller or input device (e.g., a gaze-actuated input/output device).

Moreover, the systems herein may include augmented reality (AR) or virtual reality (VR) tool(s) that can aid an operator during a surgical procedure by providing enhanced views of a surgical space (including within a body cavity of a patient) including labels and/or information about the space and the surgical instruments therein. Furthermore, the physical layout and/or configuration of the robotic surgery systems herein may improve the ergonomics (e.g., geometry, usability) between each of the robotic arm, end effector, single operator, and patient, as well as the ergonomics of the end effector disposed within the patient. In some variations, a geometry of the end effector connector may be configured to provide clearance (e.g., working space) beneath the robot for one or more of the patient, end effector, and operator, thereby increasing the efficiency of a surgical space and procedure. For example, the end effector connector may have a configuration that facilitates (e.g., improves) physical access to the end effector. Additionally, or alternatively, the robotic surgery system may position the robotic arm away from the patient and improve an accessible range of an end effector coupled to the robotic arm.

Overall, the systems and devices herein may improve an operator's experience in preparation for and during a surgical procedure, which may reduce complications during the procedure and improve patient outcomes. The systems and devices herein may be used to execute one or more methods for performing robotic surgery. In some variations, a method for performing robotic surgery may include: first, coupling an end effector connector to a coupling mechanism, the coupling mechanism coupled to a distal end of a robotic arm, where the coupling mechanism may include a cylindrical housing defining a longitudinal axis, a sleeve disposed around the cylindrical housing, an actuator disposed within the cylindrical housing, and one or more projections extendable from and retractable into a sidewall of the cylindrical housing, where the end effector connector may include an annular portion releasably coupled to the cylindrical housing, and the annular portion may include one or more grooves configured to receive the one or more projections of the cylindrical housing; second, actuating the actuator with a first hand of an operator to retract the one or more projections within the sidewall of the cylindrical housing to release the annular portion from the cylindrical housing; third, the method may include receiving the annular portion with the first hand of the operator.

Another method for performing robotic surgery may include controlling a support arm using support arm control signals that are generated by an operator using ergonomic input devices. One such method may include receiving a robotic arm control signal via actuation of a foot switch by a single foot of an operator. The foot switch activation may correspond to end effector motion via the robot arm. Motion of a robotic arm with at least three degrees of freedom may be controlled based on the received robotic arm control signal. Similarly, a method for controlling movement of an end effector coupled to a support arm may include measuring a gaze of an operator, and actuating the support arm to move the end effector based on the measured gaze. Here, an image of the end effector may be provided that includes a reference marker overlayed onto the end effector. Accordingly, the operator may observe a position of the end effector via the reference marker, even when the end effector is out of sight (e.g., within a body cavity of a patient).

Furthermore, methods that facilitate preparation of robotic surgery (e.g., end effector registration) are also provided. For example, methods for measuring surgical parameters, such as a position of an incision on the patient within the surgical space and/or a length of an end effector are provided. For example, an end effector (e.g., visualization device, endoscope) may have different lengths and diameters. A length of an end effector may be used by a robotic surgery system to set constraints on the movement of the end effector within a surgical space (e.g., relative to a patient body). However, an incorrect input or determination of an end effector length by an operator may lead to tissue damage. Accordingly, a method for determining an unknown length of an end effector may include positioning the end effector relative to a body (e.g., a patient body, a planar surface, an object or marker within a surgical space) and defining the relative positions of the end effector to determine its length via triangulation. For example, a controller may be used to accomplish these methods. In some variations, the controller may be configured to prompt an operator to position the end effector (e.g., via actuating a robotic arm coupled thereto) relative to the body, and may be configured to calculate the unknown length of the end effector by recording and using position information corresponding to the end effector. In some variations, the prompting may occur via a user interface that instructs the operator through a protocol for determining the unknown length of the end effector. Accordingly, the methods herein may aid in preparing robotic surgery by guiding an operator through a procedure for determining surgical parameters.

The systems and devices described herein may be used to perform surgical procedures such as one or more of cholecystectomy, appendectomy, colectomy, hernia repair, sleeve gastrectomy or other bariatric procedures, nephrectomy, hysterectomy, oophorectomy, lobectomy, salpingectomy, fallopian tubal ligation, and hernia repair including inguinal and hiatal. Variations of robotic surgery systems, devices, and methods, and aspects thereof, are described below.

### Systems and Devices

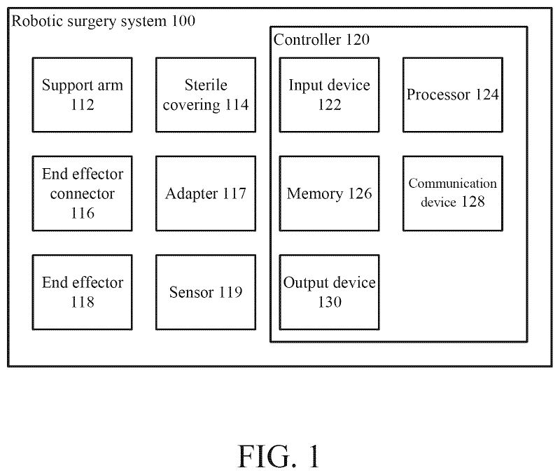

Generally, the robotic surgery systems described herein may be operated by a single operator using intuitive control schemes, improved ergonomics, and patient safety monitoring. A block diagram of an exemplary robotic surgery system 100 is depicted in FIG. 1. The system 100 may comprise one or more of a support arm 112, a sterile covering 114, an end effector connector (“connector”) 116, an end effector 118, a sensor 119, and a controller 120 with an input device 122, a processor 124, a memory 126, a communication device 128, and an output device 130, each of which are described in more detail herein. In some variations, the support arm 112 may be configured to moveably suspend, hold, and/or operate an end effector relative to a patient (e.g., patient on a patient platform) based on control (e.g., control inputs, manual manipulation) of a single operator. In some variations, the sterile covering 114 may be disposed between the support arm 112 and the end effector 116 to form a sterile field. For example, the sterile covering 114 may be disposed between a distal end of the support arm 112 (e.g., via the coupling mechanism, as described in more detail herein throughout) and a proximal end of the end effector connector 116 (e.g., via the annular portion, as described in more detail herein throughout). Coupling of the support arm 112 and end effector connector 116 may be temporary (releasable) or permanent.

In some variations, the end effector connector 116 may be configured to couple the support arm 112 to the end effector 118 and facilitate single operator operation (e.g., assembly, control, disassembly). For example, the end effector 116 may facilitate single operator control through simplified and rapid end effector exchanges as well as improved surgical procedure ergonomics that may reduce procedure times and improve patient outcomes. In some variations, the end effector connector 116 may be configured to couple to the end effector 118 via an adapter 117. The adapter 117 may comprise an outer dimension that is about equal to an inner dimension of a clamp of the end effector connector 116 such that the adapter 117 fits within the clamp. The adapter 117 may comprise a lumen configured to receive the end effector 118 (e.g., an endoscope) therethrough. Accordingly, in some variations, the end effector connector 116 may couple to the end effector 118 indirectly, via the adapter 117. In some variations, the end effector 116 may facilitate single-handed operator operation of the robotic surgery systems described herein. As described in more detail herein, the end effector 118 may comprise one or more end effectors used in a surgical procedure.

The sensor 119 may include one or more sensors configured to measure one or more characteristics corresponding to one or more of the patient and surgery system 100 including, but not limited to, one or more of the support arm 122, the sterile covering 114, the end effector connector 116, the end effector 118, and the input device 122. In some variations, the sensor 119 may include a plurality of sensors. Non-limiting examples of the sensor 119 may include: a force sensor, an accelerometer (e.g., 3-axis), gyroscope (e.g., 3-axis), a position sensor, an optical sensor, a motion sensor, a pressure sensor, and a magnetic sensor.

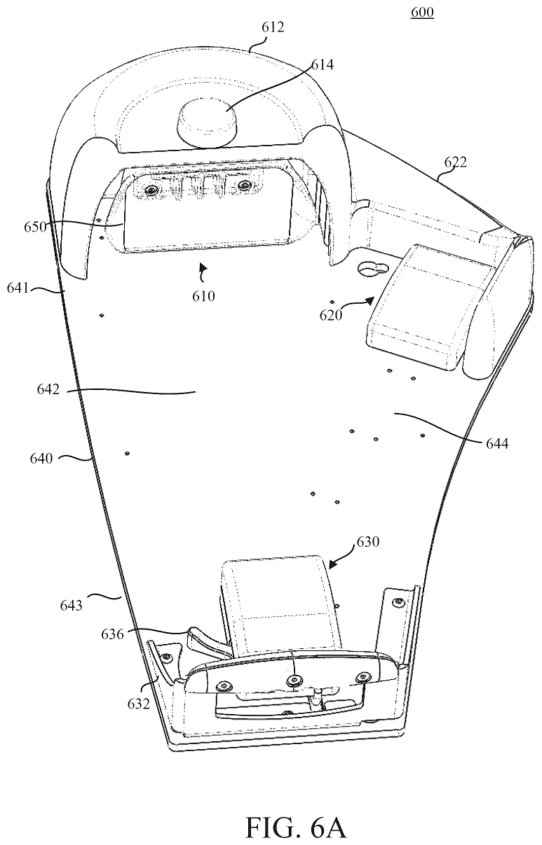

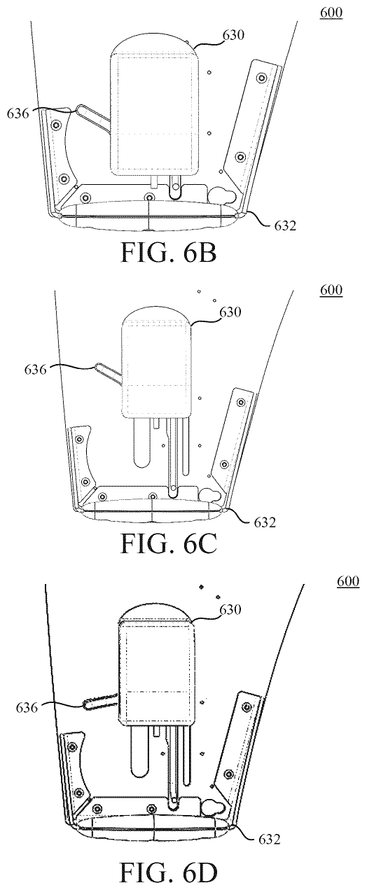

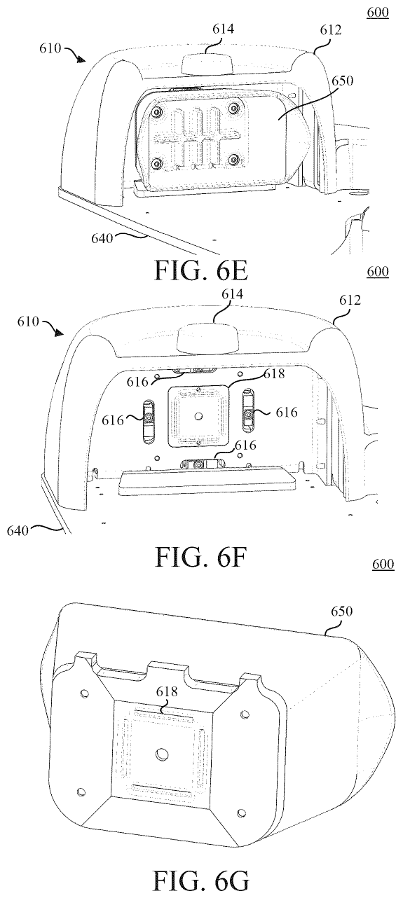

The input device 122 may be a controller configured to generate an input signal based on an operator input (described in more detail with respect to, e.g., FIGS. 6A-6G). The operator input may correspond to a force or signal detected by the input device 122, such as an applied force or a gaze of the operator. In some variations, the input device 122 may facilitate single-footed operator control of the robotic surgery systems described herein. In some variations, the processor 124 and memory 126 may be configured to control the surgery system 100. In some variations, the communication device 128 may be configured to communicate with one or more components of the system 100 as well as with networks and other computer systems. In some variations, the output device 130 may be configured to output data corresponding to the surgery system 100.

The systems described herein may allow a single operator may independently control a set of end effectors coupled to a robotic surgery system without assistance from a second operator. For example, in some variations, a robotic surgery system may include an end effector coupled to a coupling mechanism of a support arm via an end effector connector. The support arm may be controlled by an operator using an input device such as input device 600 as described herein. In some variations, the system may include a plurality of support arms coupled to a plurality of end effector connectors, where each of the plurality of support arms may be controlled by an input device.

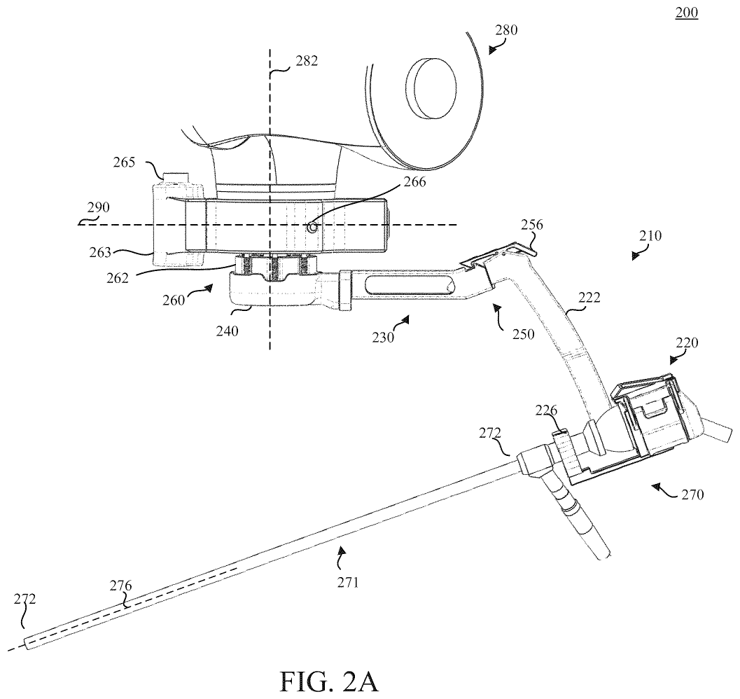

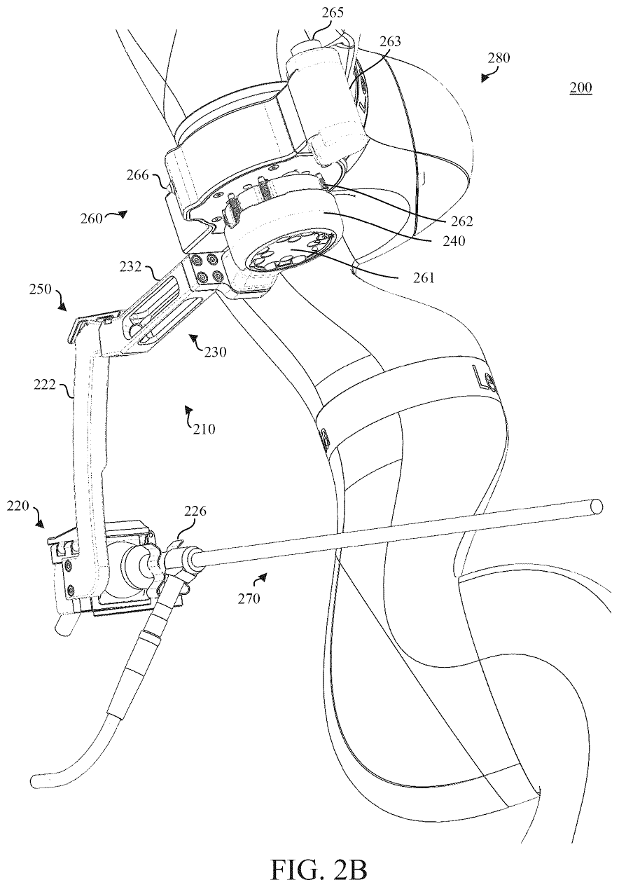

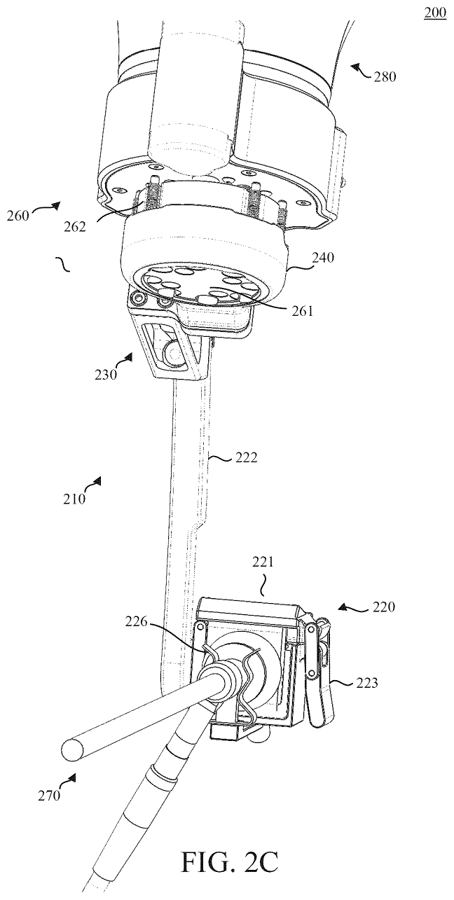

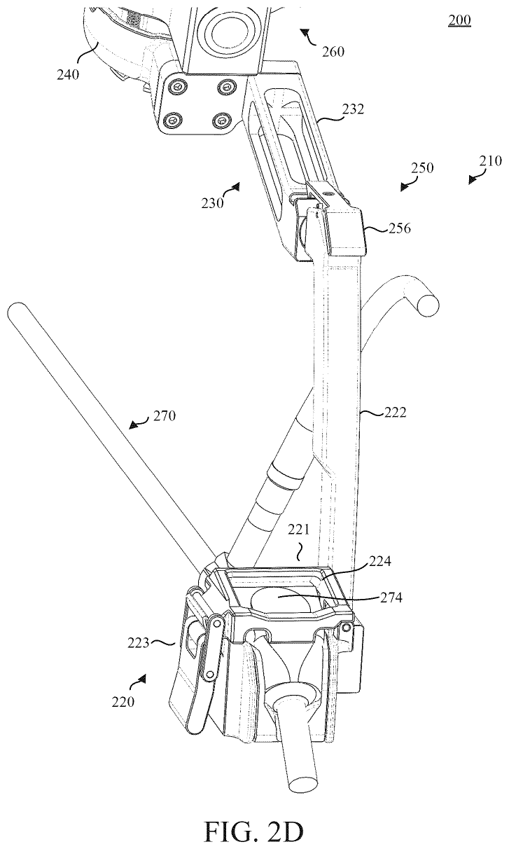

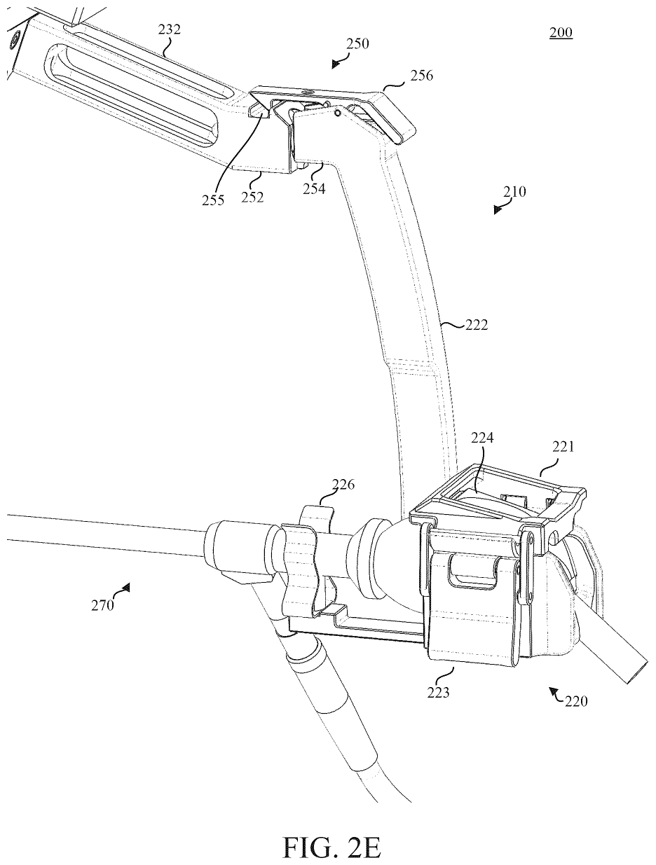

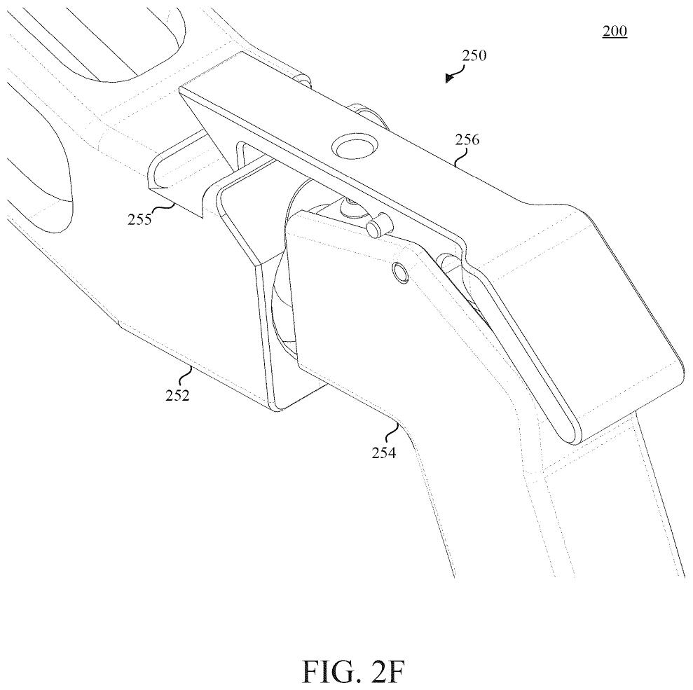

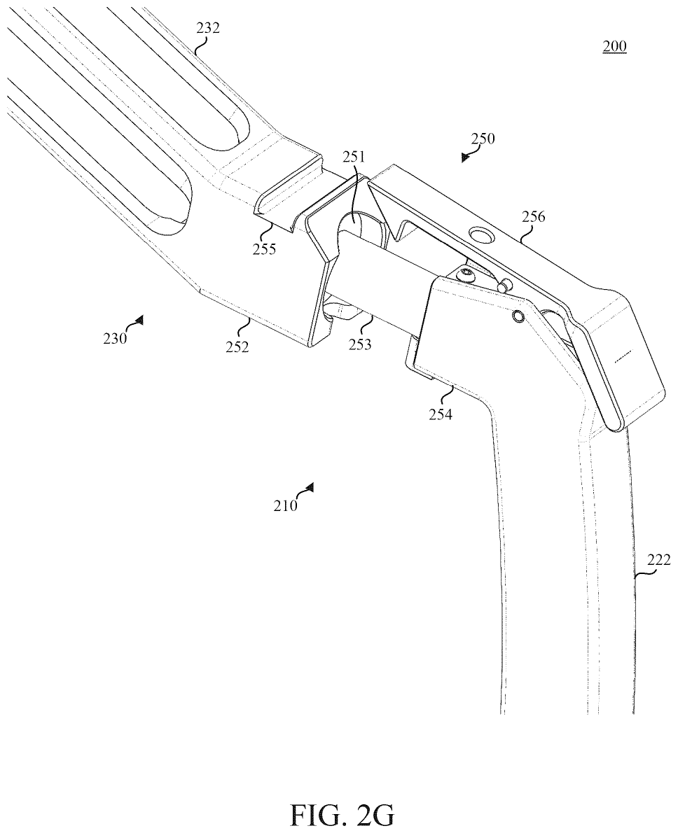

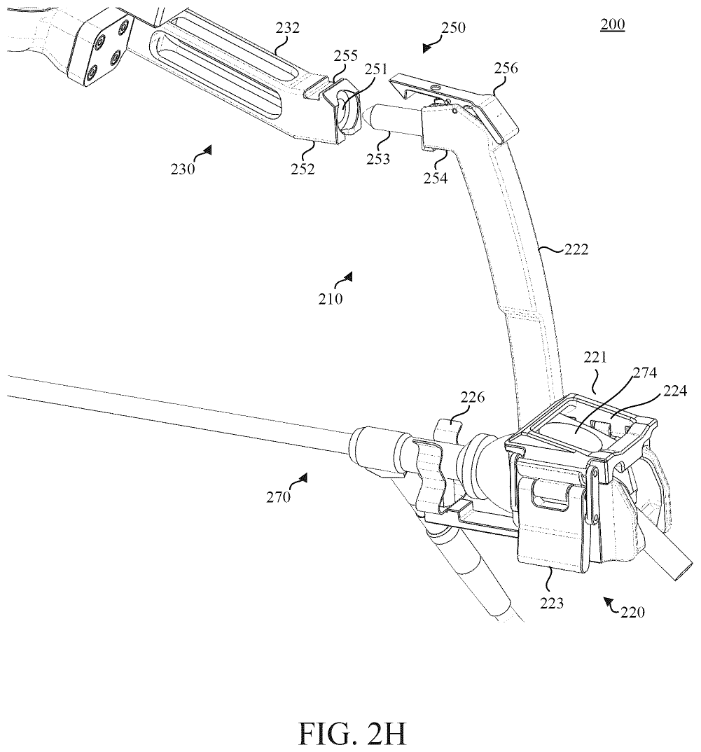

Various aspects of the systems herein will be described below with reference to FIGS. 2A-2I. FIG. 2A depicts a side view of the robotic surgery system 200. FIGS. 2B-2D depict various perspective views of the robotic surgery system 200. FIG. 2E-2H depict perspective views of the end effector housing release mechanism 250 of the robotic surgery system 200. FIG. 2I depicts a top view of the end effector housing 220 of the robotic surgery system 200. The system 200 may generally include a support arm 280 (e.g., robot, robotic arm), an end effector 270, and an end effector connector 210 (“connector”). In some variations, the system 200 may additionally include an adapter (not shown) configured to secure a length of the end effector 270 within the end effector connector 210.

### 1. End Effector

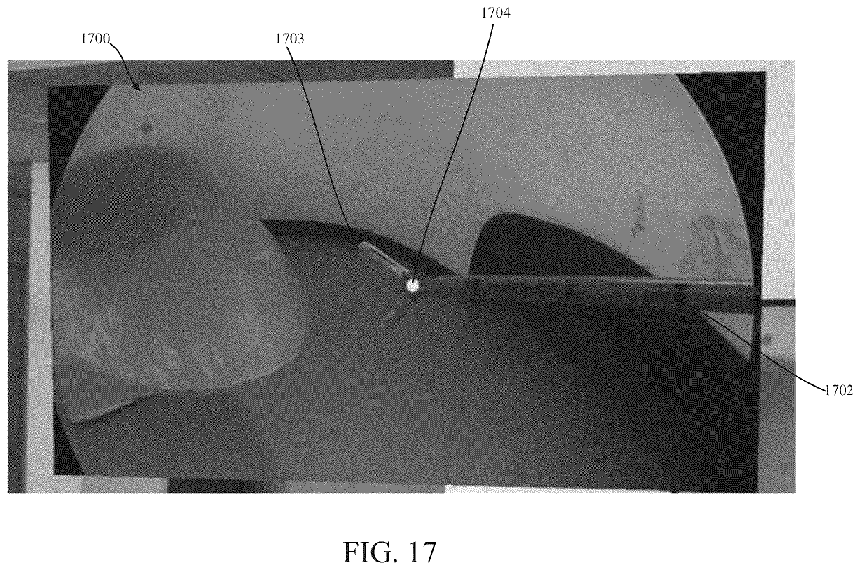

Generally, the end effectors (e.g., end effector 270 of FIG. 2A) described herein are not particularly limited and may comprise one or more of a visualization device, a grasper, a retractor, a magnetic positioning device, a sensor, an intracavity device, a delivery device, a stapler, a clip applier, an electrocautery hook, and other surgical instrument that may be advanced in a minimally invasive manner through an access site. In some variations, the end effector may comprise a magnetic portion. In some variations, the end effector may comprise an RFID tag so that a position of the end effector within a surgical space (e.g., within a body cavity of a patient) may be tracked. For example, an input/output device may be configured to receive a signal from the RFID that is indicative of the position of the end effector, and may be configured to overlay a reference marker or tag onto or adjacent to the end effector via a display so that an operator may easily view the position of the end effector throughout a surgical procedure. The reference marker may comprise one or more of a symbol, image, or text. For example, FIG. 17 shows an exemplary augmented reality (AR) image 1700 having a view of an end effector 1702 and a reference marker 1704 overlayed onto the distal (instrument) end 1703 of the end effector 1702. In some variations, the reference marker may comprise a digital reproduction, or digital outline, of the end effector that is overlayed onto or around the end effector.

In some variations, an end effector (e.g., visualization device, intracavity device) may be configured to be introduced into a body cavity or lumen through an access site such as a trocar or other suitable port, or through a natural orifice. The end effectors may be used within any suitable body cavity or lumen such as but not limited to the abdominal cavity, thoracic cavity, stomach, or intestines. The end effectors advanced into a body cavity or lumen may perform a number of functions and are described in detail herein. The end effectors advanced into the body cavity or lumen through an access site may be advanced such that the end effector does not block the introduction and/or retrieval of other end effectors using the access site. Thus, a plurality of end effectors may be disposed and actuated within a patient body cavity or lumen.

The end effectors may be configured to be attracted to one or more magnets positioned externally of the body to move, reposition, and/or hold the intracavity device (which may in turn provide traction for tissue held by or otherwise in contact with the intracavity device). Accordingly, at least a portion of the intracavity devices described herein may be formed from or otherwise include one or more metallic or magnetic materials which may be attracted to a magnetic field. The materials may include one or more magnetic or ferromagnetic materials, such as, for example, stainless steel, iron, cobalt, nickel, neodymium iron boron, samarium cobalt, alnico, ceramic ferrite, alloys thereof and/or combinations thereof. The magnetic portion of the intracavity device may thus be attracted to a magnetic field produced by an external magnetic positioning device. Furthermore, in some variations, the magnetic portion of the intracavity device may allow coupling to a delivery device, as described in more detail herein.

Referring to FIG. 2A, in some variations, the end effector 270 may comprise a body 271, having a first end or portion 272 and a second, opposite end or portion 273. As shown, the body 271 may be an elongate body. In some variations, the body 271 may comprise a cylindrical shape. The first (e.g., distal) or portion 272 may comprise an instrument (e.g., visualization device, grasper, etc.) and the second (e.g., proximal) end or portion 273 may be configured to couple to the support arm 280. In some variations, the end effector 270 (e.g., the second end or portion thereof) may be configured to couple to the support arm 280 via an adapter (not shown) and a connector 210 for the end effector, as will be described in detail herein.

In some variations, the body 271 may comprise length of about 5 cm to about 100 cm, such as about 10 cm to about 80 cm, about 15 cm to about 70 cm, about 20 cm to about 60 cm, about 25 cm to about 50 cm, or about 30 cm to about 40 cm, including all ranges and sub-values therebetween. In some variations, a width or diameter of the body 271 may be about 1 mm to about 50 mm, such as about 2.5 mm to about 40 mm, about 5 mm to about 30 mm, about 7.5 mm to about 20 mm, or about 10 mm to about 15 mm, including all ranges and sub-values therebetween. In some variations, the width or diameter of the body 271 may be about equal to or less than a width or diameter of the adapter (e.g., a lumen thereof) and/or the connector (e.g., a clamp thereof). For example, the width or diameter of the body 271 may be about equal to or less than 50 mm, such as about equal to or less than 40 mm, about equal to or less than 30 mm, about equal to or less than 25 mm, about equal to or less than 20 mm, about equal to or less than 15 mm, about equal to or less than 10 mm, or about equal to or less than 5 mm.

In some variations, a known (i.e., predetermined) length of the body 271 may be used to determine a position of parameter, such as a reference point between the end effector 270 and a surface of a body or object within a surgical space (e.g., a patient body, surgical table, marker, etc.). For example, the length of the body 271 may be input into a user interface and used during a triangulation process to determine the position of the reference point, as described in further detail herein. Moreover, in some variations, an unknown length of the body 271 may be determined via a triangulation protocol that is described with reference to method 2200 of FIG. 22.

In some variations, an end effector may comprise a visualization device (e.g., endoscope) configured to be visualize a desired field of view during a minimally invasive procedure. In some variations, an end effector may comprise a grasper used to grasp, retract or otherwise provide remote manipulation and/or traction to tissue. In particular, magnetically controlled graspers may be advanced into a patient and releasably engage tissue. Graspers suitable for use in the surgery systems here are described in U.S. patent application Ser. No. 14/019,370, filed Sep. 5, 2013, and titled “Grasper with Magnetically-Controlled Positioning,” U.S. patent application Ser. No. 15/195,898, filed Jun. 28, 2016, and titled “Laparoscopic Graspers and Systems Therefor,” U.S. patent application Ser. No. 13/132,185, filed Aug. 17, 2011, and titled “Remote Traction and Guidance Systems for Mini-Invasive Surgery,” and International Patent Application No. PCT/US2016/027390, filed Apr. 13, 2016, and titled “Grasper with Magnetically-Controlled Positioning,” each of which is hereby incorporated by reference in its entirety.

In some variations, an end effector may comprise a retractor described used to retract or otherwise support and/or move internal organs of a patient. In particular, magnetically controlled retractors may be advanced into a patient and retract tissue to displace it from a surgical site inside the patient and/or otherwise engage tissue to increase surgical access to that tissue. Furthermore, the retractors may be configured to be maintained in position without requiring a handle or grasper. For example, in some variations, a retractor may be configured to form a sling to retract tissue. The terminal ends may comprise a magnetic material or have magnetic masses disposed on them, such that they are configured to be attracted to a magnetic field. When a portion of the retractor is looped underneath a portion of tissue, at least a portion of the tissue may be suspended by the retractor and moved towards the patient wall. In some variations, the retractor may be configured to transition between a substantially linear configuration and the curvilinear configuration.

Other retractors suitable for use in the surgery systems here are described in International Patent Application No. PCT/US2016/027385, filed Apr. 13, 2016, and titled “Retractor Systems, Devices, and Methods for Use,” which is hereby incorporated by reference in its entirety. Other suitable retractors may include, for example, one or more of a coiled retractor, cradle retractor, lever retractor, platform retractor, and J-hook.

### 2. End Effector Connector

Generally, the end effector connectors (“connectors”) described herein may be configured to releasably connect an end effector to a support arm to facilitate rapid single operator operation and/or exchange of an end effector coupled to a support arm, thereby enabling single operator operation without a second operator to improve operator ergonomics and reduce sterile field management and procedure times. In some variations, the single operator operation may be single-handed operator operation, further improving operator ergonomics and reducing sterile field management and procedure complexity.

Accordingly, the end effector connector 210 may comprise a first end configured to releasably couple to the support arm 280 and a second (e.g., opposite) end configured to releasable couple to the end effector. In some variations, the first end may be a proximal end and the second end may be a distal end. In some variations, the end effector connector 210 may be configured to releasably couple the end effector 270 to a distal end of the support arm 280. The end effector connector 210 may comprise an arm 230 and housing release mechanism 250 at the first end, a clamp 226 and housing 220 at or toward the second end, and a handle 222 coupling the first and second ends of the connector 210. The shape and dimensions of the end effector connector 210 may control the positioning and/or orientation of the end effector 270 relative to the support arm 280. For example, as shown in FIG. 2A, a longitudinal axis 276 of the end effector 270 may be angled at a non-perpendicular (e.g., oblique) angle relative to a longitudinal axis 282 of the support arm 280 in order to provide a favorable entry angle for an end effector 270 used in a surgical procedure. For example, an angle between a longitudinal axis 282 of the support arm 280 and a longitudinal axis 276 of the end effector may be between about 90 degrees and about 130 degrees or between about 105 degrees and about 125 degrees, including all ranges and sub-values in-between.

Moreover, a space directly below the support arm 280 (e.g., along the longitudinal axis 282) may comprise empty space absent the end effector connector 210 and the end effector 270, which may be reserved for patient anatomy (e.g., patient abdomen). This space reservation (e.g., clearance) formed by the end effector connector 210 may improve the ergonomics and safety of a surgical procedure. For example, as shown in FIG. 2A, the end effector 270 may be located below and away from the support arm 280. With respect to FIG. 2A, a patient may be disposed under and/or to the left of the support arm 280 while the end effector 270 may be disposed under and/or to the right of the support arm 280.

Each component of the end effector connector 210, including the housing 220, the clamp 226, the housing release mechanism 250, and the arm 230 is described in detail below.

### 2.1. Housing

In some variations, the housing 220 of the end effector connector 210 may be configured to receive the end effector 270. For example, the housing 220 may be configured to hold the end effector 270 in a predetermined position and/or orientation relative to the support arm 280. As depicted in FIGS. 2C-2E and 2H-2I, the housing 220 may hold the end effector 270 via top portion 221 and clip 223. The top portion 221 may be hinged and configured to be rotated from an open position (to receive the end effector 270) to a closed position (to grip the end effector 270). The clip 223 may also be a hinged component that is configured to mechanically couple to (e.g., via press fit) a side of the top portion 221 to maintain a position of the end effector 270 within the housing 220. A distal portion of the clip 223, distal portion 225, may facilitate release of the clip 223 from the top portion 221. For example, an operator may press the distal portion 225 to release the clip 223 from the side of the top portion 221, thereby allowing the top portion 221 to be rotated to the open position so that the end effector connector may be removed from (or otherwise adjusted within) the housing 220. In some variations, the housing 220 may comprise a housing handle 222 configured to be held and/or manipulated by an operator (e.g., using a single hand). For example, the operator may grip the housing handle 222 with a single hand to manipulate the position and/or orientation of the end effector 270. While the support arm 280 may generally be controlled by a processor to position the end effector 270 as desired, manual manipulation by the operator holding the housing handle 222 may facilitate fine adjustments to the position and/or orientation of the end effector 270.

In FIGS. 2A-2D, the housing 220 is illustratively configured to receive an endoscope 270, although any end effector may be used. In some variations, the housing 220 may be configured to facilitate operator access to the end effector 270. As shown in FIG. 2D, the top portion 221 of the housing 220 may define an aperture 224 (e.g., cutout) configured to facilitate access to end effector controls such as an input device 274 (e.g., endoscope or camera control button(s), switch). Accordingly, the housing 220 and the end effector connector 210 may function unobtrusively to the operator. Furthermore, the housing 220 may improve the ergonomics of end effector 270 manipulation by providing a handle 222.

### 2.2. Clamp

The clamp 226 may be configured to secure a position of the end effector 270 when it is coupled to the connector 210. Put another way, the clamp 226 may be configured to apply a stabilizing force to the end effector 270 to maintain its position relative to the connector 210. To do so, the clamp 226 may be configured to receive a portion or length of the end effector 270 therein, such as within a recess or lumen thereof. The clamp 226 may be configured to completely or at least partially surround the length end effector 270 therein. In some variations, the clamp 226 may receive the end effector 270 indirectly, such as via an adapter (not shown), which will be described in detail below. Generally, the adapter may be used with the end effector 270 so that the combined adapter and end effector may have a width or diameter that is about equal to or less than the width or diameter of the recess or lumen of the clamp 226. Thus, the adapter may enable the clamp 226 to secure the end effector 270 therein.

The clamps herein may comprise any suitable clamp, such as a clip (e.g., clamp 226), an over-center clamp, an Irwin clamp, a tri-bearing clamp, combinations thereof, and the like. In some variations, the clamps herein may comprise a plurality of clamps, such as at least two clamps of a same or different type. For example, the clamps may include a first clamp (e.g., a clip, such as clamp 226) and a second clamp of a different type (e.g., an over-center clamp).

In some variations, the clamps herein may comprise a base. The base may be indirectly or directly secured to the handle (e.g., handle 222). In some variations, a at least a portion of a perimeter of the recess or lumen of the clamp may be defined by the base. In some variations, the clamp may further comprise a moveable portion (e.g., at least one moveable portion) that is coupled to the base (e.g., via hinges). In some variations, the base may comprise a first recess configured to receive or surround a first portion of the end effector and/or adapter, and the moveable portion may comprise a second recess configured to receive or surround a second portion of the end effector and/or adapter. In some variations, the first and second recesses may comprise a same or mirrored shape (e.g., semicircle, half circle, half hexagon, V-shape, U-shape, etc.). In some variations, the first recess may comprise a first shape (e.g., V-shape or a semicircle) and the second recess may comprise a second, different shape (e.g., a semicircle or a V-shape).

In some variations, the first and second recesses may comprise a same width or diameter of about 1 mm to about 50 mm, such as about 2.5 mm to about 40 mm, about 5 mm to about 30 mm, about 7.5 mm to about 20 mm, or about 10 mm to about 15 mm, including all ranges and sub-values therebetween. In some variations, the first and second portions of the end effector 270 may be opposing cross-sectional portions its body (e.g., body 271).

In some variations, the movable portion may be movable (e.g., rotatable) relative to the base. The relative positions of the movable portion and the base may define open and closed configurations of the clamp. For example, in the open configuration, the longitudinal axes of the movable portion and the base may form an angle that is greater than about 0 degrees and less than or equal to about 90 degrees, or greater than about 0 degrees and less than or equal to about 180 degrees. In this configuration, the moveable portion may uncover the base, revealing the recess thereof and facilitating positioning of the end effector (and/or the adapter) therein. Conversely, in the closed configuration, the longitudinal axes of the movable portion and the base may form an angle of about 0 degrees. Here, the moveable portion may be covering the base, thereby allowing the end effector 270 (and/or the adapter) to be held between the first recess of the base and the second recess of the movable portion.

In some variations, the clamp 226 may further comprise a lock or actuator (not shown). The lock or actuator may be configured to maintain the clamp in the closed position (or the open position). The lock may be coupled (e.g., rotatably) to one or both of the base and the moveable portion of the clamp 226. In some variations, a first end of the lock (e.g., a free end not coupled to the base or movable portion) may be configured to releasably engage an aperture within the base of the clamp 226 o maintain the clamp in the closed configuration. For example, a distal end of a body of the lock may be configured to releasably engage the aperture. As another example, in some variations, a projection on the distal end of the body of the lock may be configured to releasably engage the aperture. In some variations, the lock body may comprise an elongate lock body.

In some variations, the clamps herein may comprise more than one movable portion, such as two, three, four, five, or more than five moveable portions. A plurality of movable portions of a clamp may be coupled (e.g., rotatably) to one or more other elements of the clamp in order to limit the movement of the one or more elements when actuating the clamp between the open and closed configurations.

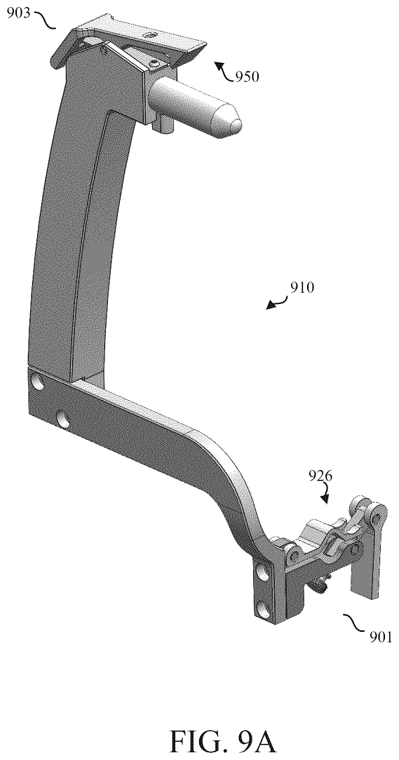

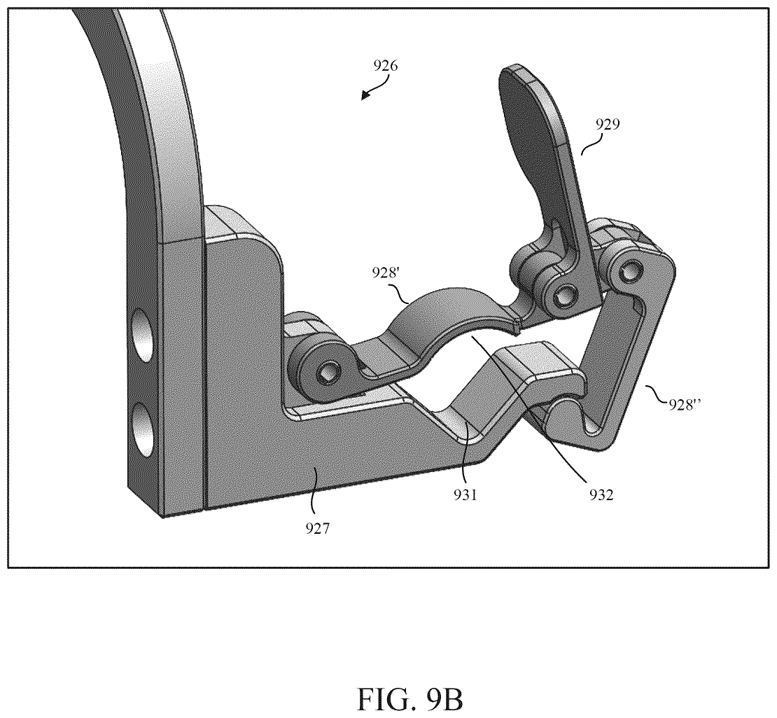

FIGS. 9A-12 depict various exemplary variations of the clamp. FIG. 9A depicts a perspective view of an end effector connector 910 having a clamp 926 situated a first end 901 of the connector 910. The clamp 926 may comprise an over-center clamp. A housing release mechanism 950 is provided at a second, opposite end 903 of the connector 910. A handle 922 extends between the clamp 926 and the housing release mechanism 950 to connect the two elements. FIG. 9B depicts a perspective view of the clamp 926 shown in FIG. 9A. As shown, the clamp 926 may comprise a base 927, a first movable portion 928′, a second movable portion 928″, and a lock 929 configured to actuate the movable portion 928 relative to the base 927 to transition the clamp 926 between open and closed configurations. In the closed position, the lock 929 may be rotated toward the first moveable portion 928′ such that it covers the moveable portion 928′. Here, a longitudinal axis of the lock 929 may form an angle of about zero or substantially zero with a longitudinal axis of the first moveable portion 928′. To transition to the open position, the lock 929 may be rotated away from the first moveable portion 928′ such that it uncovers the moveable portion 928′. The second moveable portion 928″ may couple the lock 929 to the base 927 (e.g., via hinges) to limit the rotational freedom of the first moveable portion 928′ and the lock 929. Moreover, the base 927 may comprise a first recess 931 and the first moveable portion 928′ may comprise a second recess 932. The first and second recesses 931, 932 may be opposite each other such that an end effector and/or adapter may be secured therebetween. Here, the first recess may comprise a first shape (e.g., a semicircle or half circle) and the second recess may comprise a second, different shape (e.g., a V-shape).

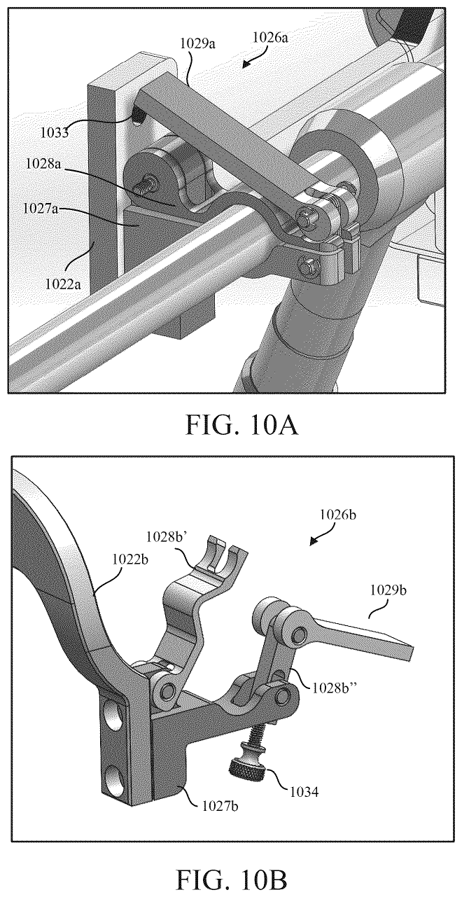

FIGS. 10A and 10B depict two perspective views of another variation of a clamp, clamp 1026a, 1026b. Like the clamp 926, the clamps 1026a, 1026b may comprise over-center clamps due to their locks 1029a, 1029b being configured to cover their respective moveable portions 1028a, 1028b′ in the closed configuration, which is shown in FIG. 10A, a longitudinal axis of the locks 1029a, 1029b may form an angle a longitudinal axis of the moveable portion 1028a, 1028b that is greater than zero. To maintain the locks 1029a, 1029b in the closed position, the bases 1027a, 1027b (or the handles 1022a, 1022b) may comprise an aperture 1033 configured to receive a portion (e.g., a distal end) of the lock 1029a, 1029b therein. To maintain the lock 1029b in the closed position, the handle 1022 of the connector 1010 may comprise an aperture (not shown) configured to receive a portion (e.g., a distal end) of the lock 1029b therein. The clamp 1026b may further comprise a second movable portion 1028b″ that is rotatably coupled to the base 1027b and lock 1029b. The second movable portion 1028b″ may be configured to receive an actuator 1034 (e.g., a screw) configured to be adjusted to correspondingly adjust a rotational freedom of the second movable portion 1028b″. For example, adjusting the actuator 1034 may comprise reducing a rotational freedom of the second movable portion 1028b″ to correspondingly reduce movement of the lock 1029b and first movable portion 1028b′.

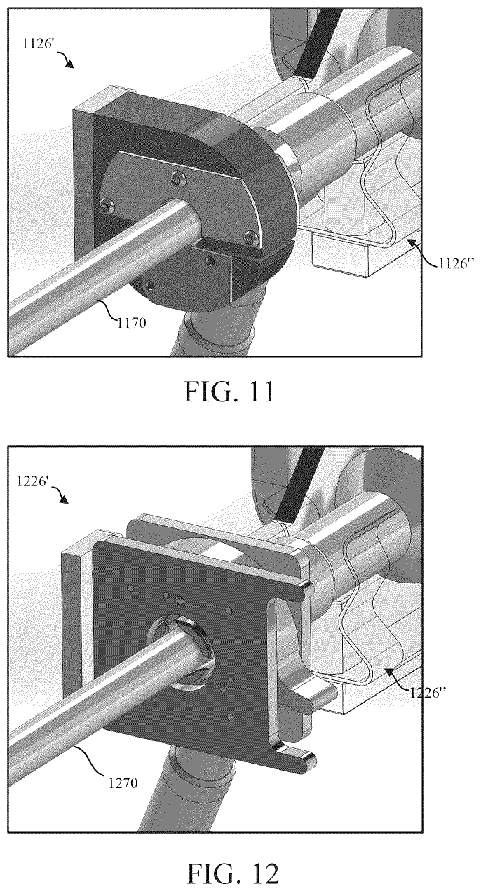

Further, FIG. 11 is a perspective view of a clamp 1126′, which may comprise a tri-bearing clamp, and FIG. 12 is a perspective view of a clamp 1226′ for use with the systems herein, which may comprise a tri-bearing clamp. In some variations, one or both of the clamps 1126′, 1226′ may be a first clamp 1126′, 1226′ that may be utilized along with a second clamp 1126″, 1226″. The second clamps 1126″, 1226″ may each be configured as a clip configured to stabilize and support more proximal portions or lengths of the end effectors 1170, 1270. This clamp redundancy may provide failure protection in the event that clamps 1126′, 1226′ are not properly secured about the end effectors 1170, 1270.

### 2.3. Arm

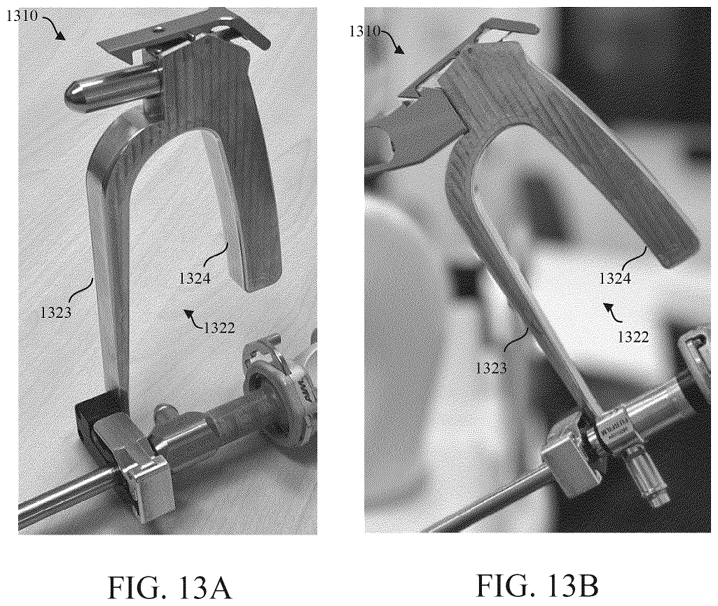

The end effector connector 210 may include an arm 230. In some variations, the arm 230 may be coupled to the annular portion 240 at a first (e.g., proximal) end, and may be releasably coupled to the handle 222 of the housing 220 via the housing release mechanism 250 at a second (e.g., distal) end. In some variations, the handle 222 may comprise a grip configured to be held in a hand of an operator to facilitate connector exchanges and connector movement (e.g., transportation within a surgical space or medical establishment). For example, turning briefly to FIGS. 13A and 13B, two perspective views of an exemplary variation of a connector 1310 are provided. As shown, the connector 1310 may include a handle 1322 with a coupling body 1323 and a handpiece 1324. The coupling body 1323 may be an elongate handle body that couples first and second ends of the connector 1310 (e.g., via the clamp 1326 and the housing release mechanism 1350). The handpiece 1324 may be situated proximally to the coupling body 1323, and may be integrally formed therewith. In some variations, the handpiece 1324 may comprise a grip, such as a coating, roughened surface, or surface pattern, to aid in grasping the handpiece 1324. The handpiece 1324 may comprise any suitable cross-sectional shape, such as a circular, rectangular, triangular, ovular, or hexagonal cross-sectional shape. A width or diameter of the handpiece 1324 may be constant or varied along a longitudinal axis of the handpiece 1324.

Referring again to FIGS. 2A-2I, in some variations, the arm 230 may include an arm handle 232. The arm handle 232 may be configured to be held (e.g., gripped circumferentially) and/or manipulated by an operator (e.g., using a single hand) in a manner similar to housing handle 222. For example, an operator may reach through one or more apertures defined by the arm handle 232 to manipulate a position of the robotic surgery system 200 relative to a surgical space or patient. In some variations, the handle 232 may allow for single-handed operator manipulation of the position of the robotic surgery system. Thus, the arm 230 may improve the ergonomics of end effector 270 manipulation.

### 2.4. Annular Portion

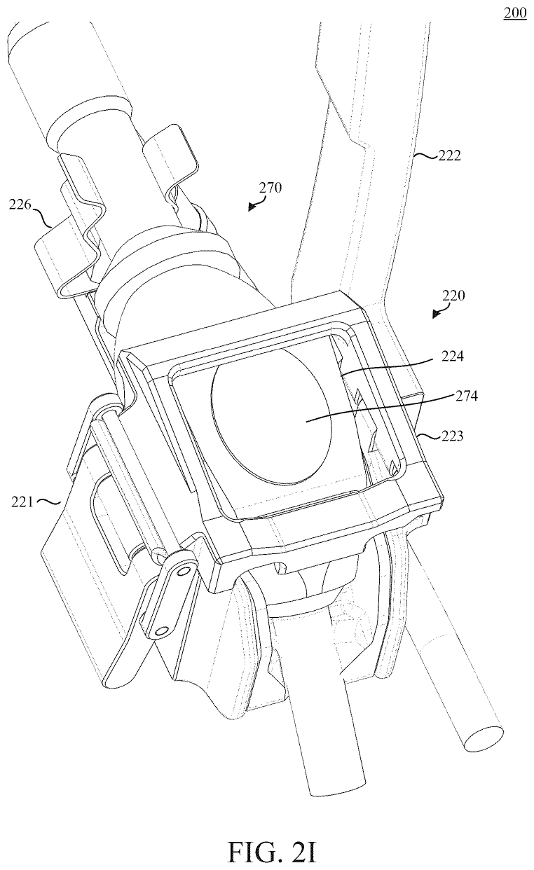

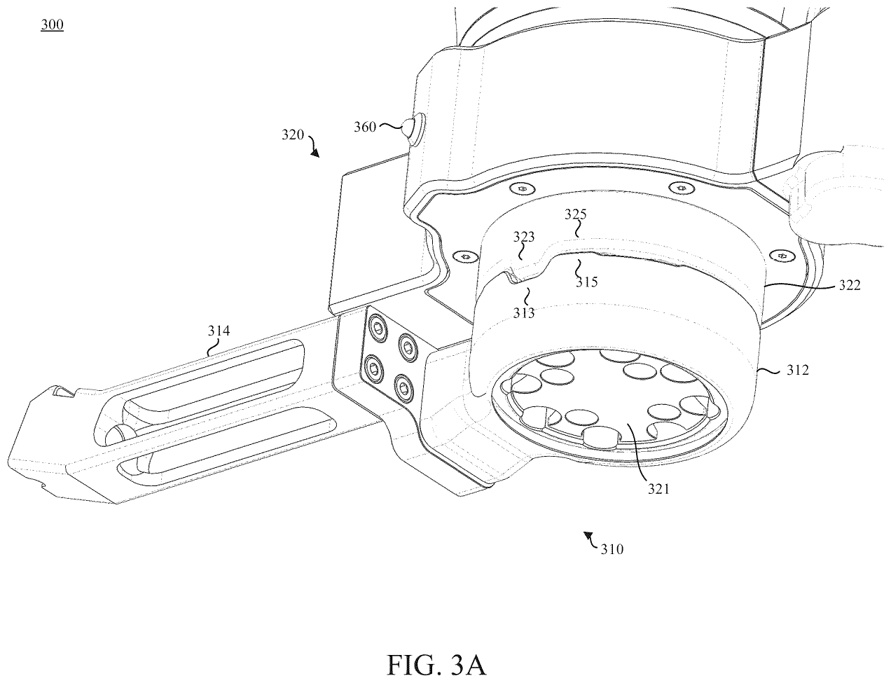

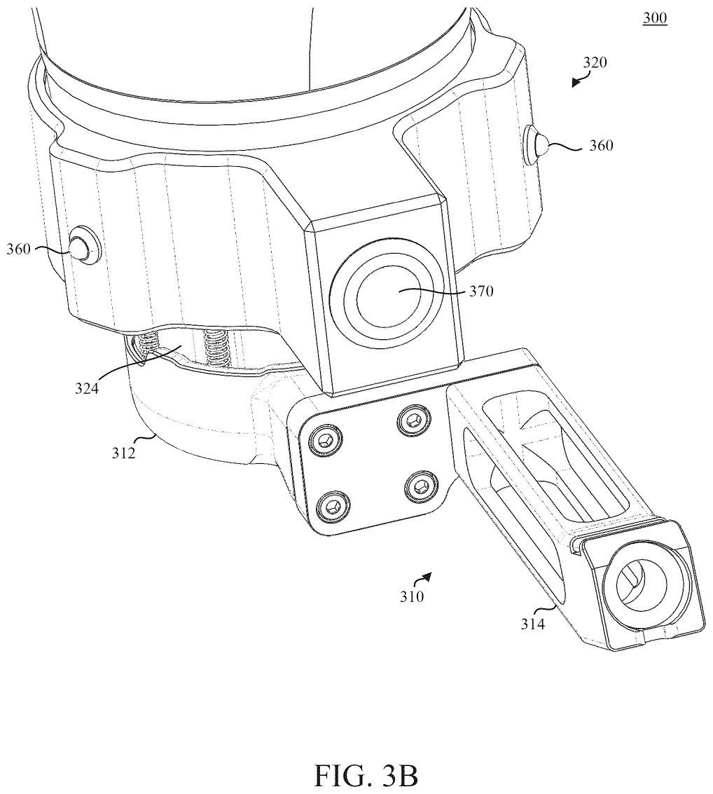

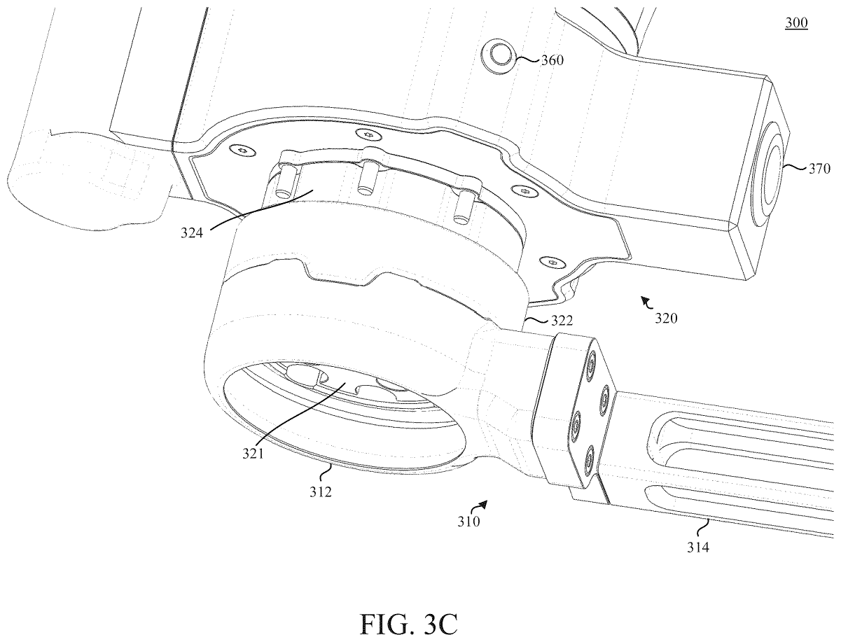

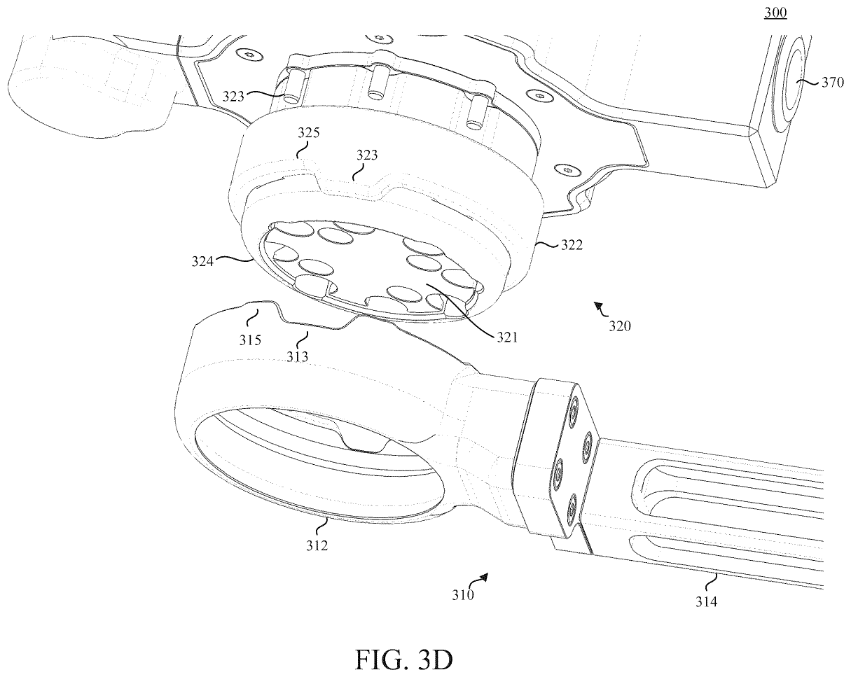

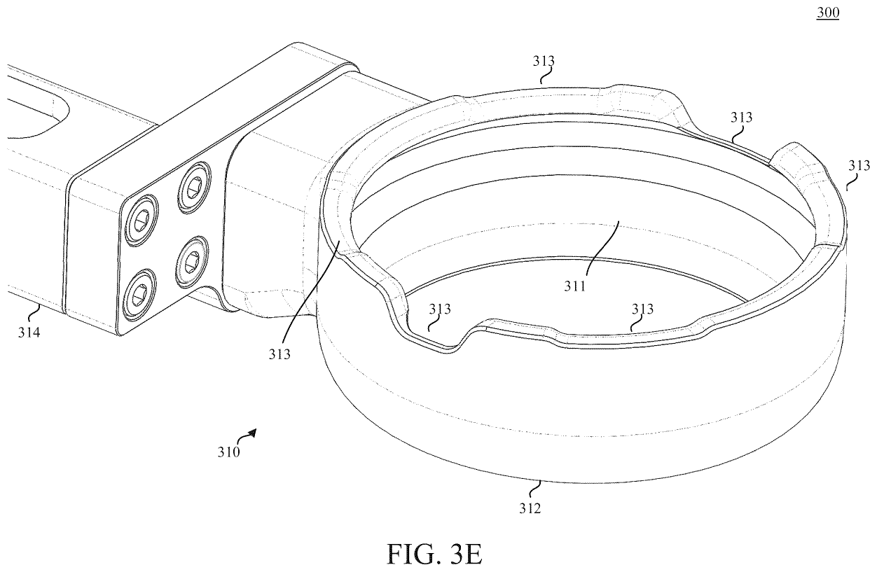

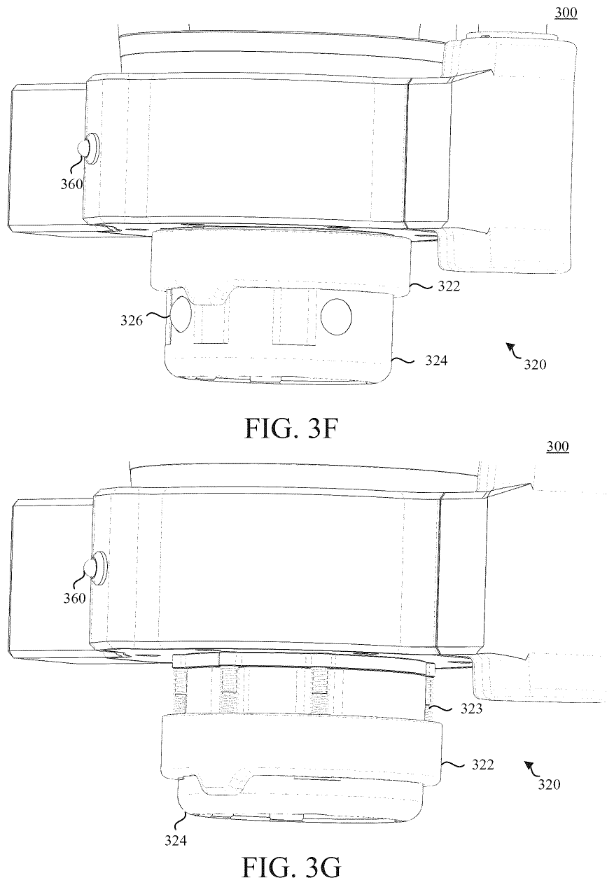

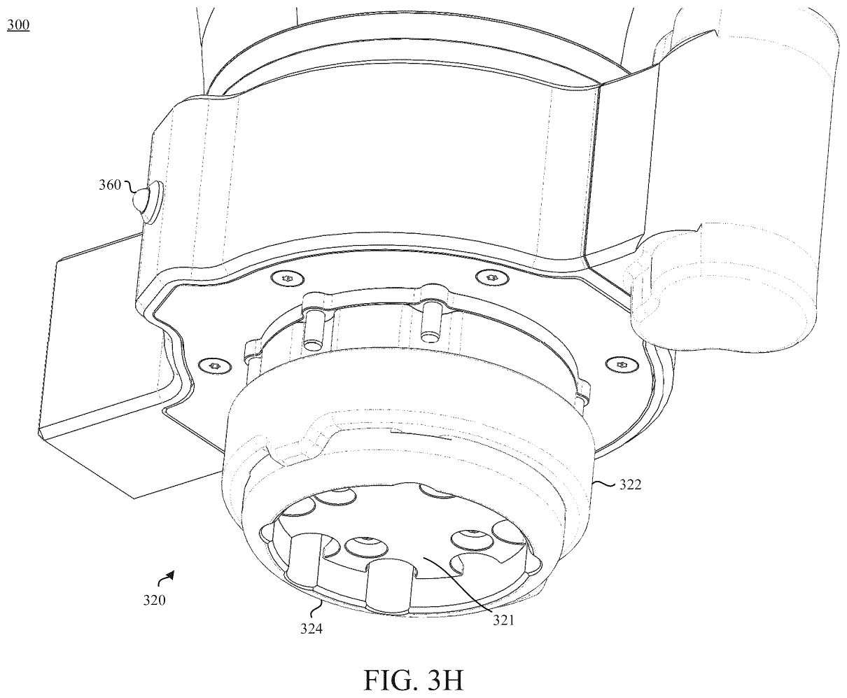

The annular portion 240 may directly couple the connector 210 to the support atm 280. In particular, the annular portion 240 may be configured to mechanically couple to: (1) the arm 230, (2) the arm 230 and the housing 220 via an engaged housing release mechanism 250, or (3) the arm 230, the housing 220, and the end effector 270 to a coupling mechanism 260 of the support arm 280. The annular portion 240 may define a central chamber and may have an interior circumference that is about equal to or greater than an exterior circumference of the cylindrical housing 262 of the coupling mechanism 260. Thus, the annular portion 240 may be configured to surround at least a portion of the cylindrical housing 262 when coupled to the support arm 280 via the coupling mechanism 260. In order to better illustrate the cylindrical housing 262 underneath a sleeve of the coupling mechanism, the sleeve is not shown in FIGS. 2A-2D, and 3B. However, a sleeve similar to as depicted in FIG. 3A, 3C, 3D, 3F-3H, and 5B-5D (e.g., sleeve 322, 422, 562), is disposed around the cylindrical housing 262.

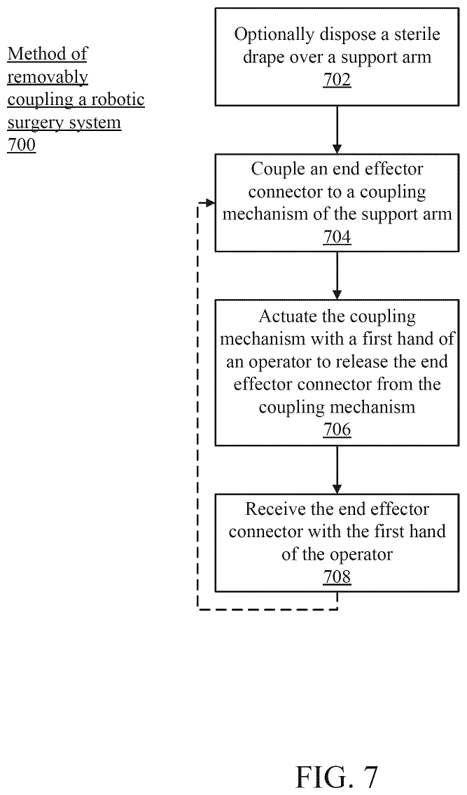

The geometry of the annular portion 240 may allow an operator to receive, directly onto a hand of the operator, the end effector connector 210 via the annular portion 240 upon release or ejection of the end effector connector 210 from the support arm 280. For example, as described in detail with respect to method 700 of FIG. 7, an operator may use a first hand to actuate a release mechanism (e.g., the actuator 261) of the coupling mechanism 260 through the central chamber of the annular portion 240 and subsequently collect (e.g., catch) the annular portion 240 (and all remaining attached end effector components and end effectors, such as one or more of the arm 230, the housing release mechanism 250, the handle 222, the housing 220, and the end effector 270) using the same first hand. That is, the annular portion 240 may be configured to be received about an actuating hand of an operator without requiring additional motions or actions of the operator following actuation of a release mechanism of the coupling mechanism 260.

Moreover, a size of the annular portion 240 may allow an operator single-handedly couple the annular portion 240 to the support arm 280 and/or otherwise single-handedly removably assemble the robotic surgery system 200 via the annular portion 240. For example, a diameter of the annular portion 240 may be about equal to an average diameter of an adult human hand, such as between about 5 in and about 10 in, between about 6 in and about 8 in, or between about 6.5 in and about 7.5 in. Thus, the diameter of the annular portion 240 may be equal to a diameter of the hand of the operator).

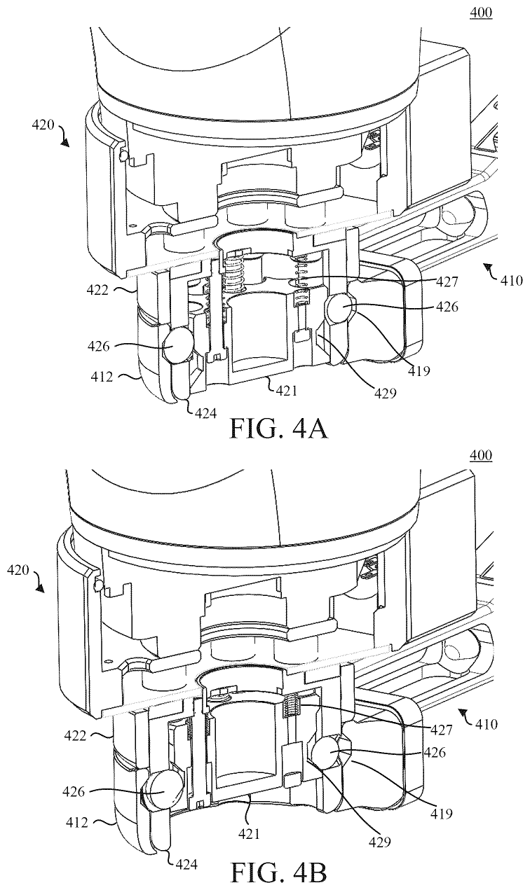

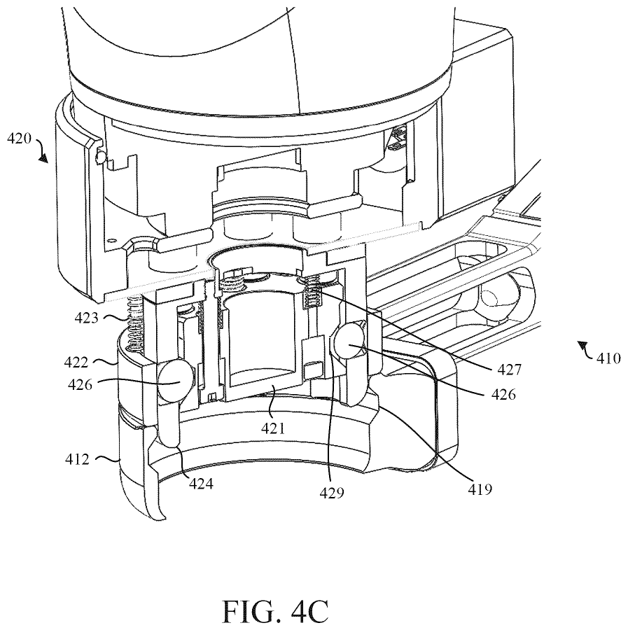

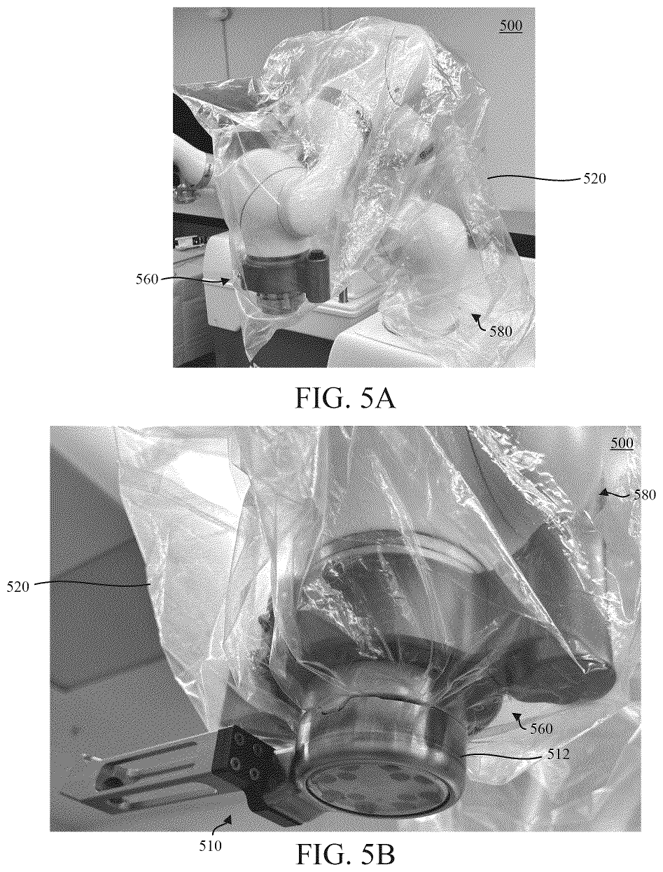

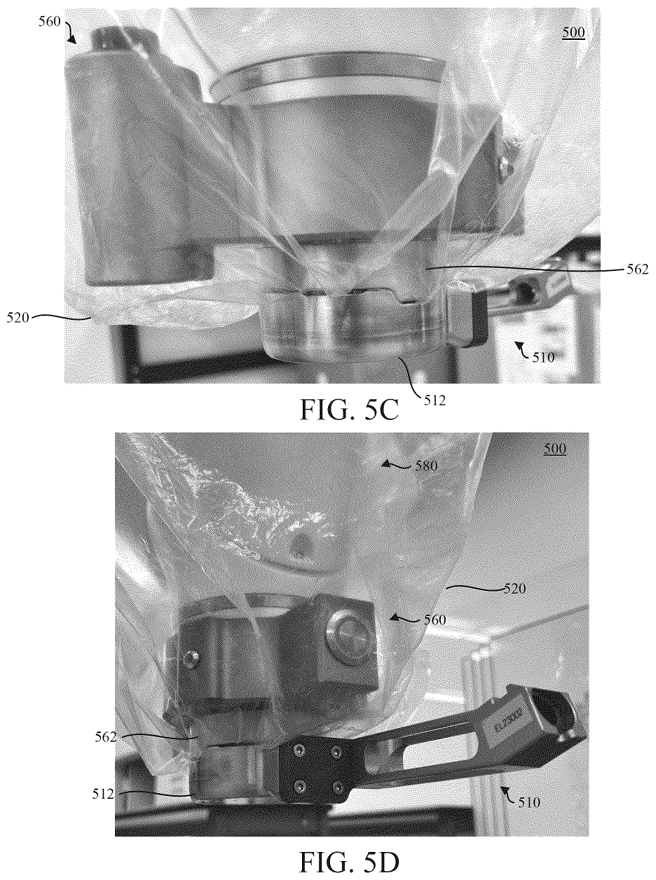

As described below with respect to FIGS. 5A-5D, in some variations, the annular portion 240 may be used to maintain a sterile drape 290 (illustrated schematically only in FIG. 2A for the sake of clarity) in position between the annular portion 240 and the support arm 280. That is, the sterile drape 290 may be coupled between the annular portion 240 and the support arm 280 such that a first side of the sterile drape 290 faces the annular portion 240 and a second side of the sterile drape 290 faces the support arm 280. In some variations, the coupling mechanism 260 may be coupled to a distal end of the support arm 280. Accordingly, the sterile drape may be disposed between a distal end of the coupling mechanism 260 of the support arm 280 and a proximal end of the annular portion 240. The annular portion 240 may be releasably coupled to the coupling mechanism 260 of the support arm 280, as described in more detail herein with respect to FIGS. 3A-4C.

### 2.5. Housing Release Mechanism

The end effector connectors herein may be configured such that a single operator may single-handedly attach and detach an end effector from a support arm within a sterile field and without affecting the sterile field. For example, referring again to FIGS. 2A-2H, the housing release mechanism 250 (which may be referred to herein as “housing attachment mechanism” or “housing release/attachment mechanism”) may be configured to releasably attach the housing 220 comprising the end effector 270 to the arm 230 (e.g., via mechanical coupling). After releasing the housing 220 from the arm 230, the arm 230 may remain coupled to the support arm 280 (e.g., via the annular portion 240 coupled to the coupling mechanism 260) to maintain the sterile field, and the end effector 270 may remain coupled to the housing 220 and so that it may be separately manipulated. This may facilitate rapid end effector cleanings and port changes.

Rapid end effector exchanges within the sterile filed that maintain sterility may reduce operator burden and surgical procedure times. In some variations, the housing release mechanism 250 may comprise a first portion 252, a second portion 254, and a switch 256 (e.g., trigger, release). The first portion 252 may be a proximal end of the arm 230, and the second portion 254 may be at a distal end of the end effector housing handle 222. In some variations, the first portion 252 may include a groove 255 configured to receive a distal end of the switch 256 (e.g., a complementary ridged end). Additionally, or alternatively, the first portion may include an aperture 251 configured to receive a complementary projection 253 of the second portion 254. Accordingly, the housing release mechanism 250 may include exterior and/or interior mechanisms for releasably coupling the end effector 270 to the support arm 280. For example, FIGS. 2F-2G depict a progression of releasing the second portion 254 from the first portion 252 such that the end effector 270 is decoupled (via end effector connector 210) from the support arm 280. To perform this action, an operator may grip the housing handle 222 with a first hand to hold the housing 220 and end effector 270 while simultaneously using a finger or thumb of the first hand to actuate the switch 256 to release the first portion 252 from the second portion 254. Actuating the switch 256 may release a distal end of the switch from the groove 255 of the first portion. The operator may then move (e.g., pull) the housing 220 and end effector 270 in a direction away from the support arm 280 (and patient) using the first hand to complete the release of the end effector 270 from the support arm 280. Repositioning the housing 220 via handle 222 may remove projection 253 of the second portion 254 from the aperture 251 of the first portion 252. Safety of the patient during an end effector change may be ensured by withdrawing the end effector 270 in a direction out of and away from the patient (e.g., parallel to a longitudinal axis of the end effector 270). Furthermore, the use of a single hand of the single operator may be more efficient than having a second operator assist. For example, a second operator provided to either press a switch or hold the handle would be redundant and would instead crowd (e.g., reduce the freedom of movement) of the first operator.

### 3. Adapter

As discussed briefly above, the systems herein may comprise an adapter to facilitate coupling an end effector to a clamp of an end effector connector (“connector”). In particular, the adapters herein may be configured to surround at least a portion of an end effector to yield a combined adapter and end effector that has a width or diameter (along the portion of the end effector surrounded) that is greater than a width or diameter of the end effector body. In this manner, an end effector with a width or diameter that is less than about equal to a width or diameter of a recess of the clamp can still be secured within the clamp because the adapter increases the size of the end effector where it attaches to the end effector. In some variations, the systems herein may comprise one or more adapters, such as a plurality thereof, each configured to fit around at least one size of end effector.

In some variations, one or more portions of the end effector that attach to the adapter may comprise a portion toward a proximal end of the end effector. A length of the portion of the end effector within the adapter may be determined by the length of the adapter (e.g., may be equal to this length). In some variations, the length of the adapter may be about equal to a width of the recess(es) of the clamp. In some variations, the length of the adapter may be greater than the width of the recess(es) of the clamp. In some variations, a length of the adapter may be about 5 mm to about 50 mm, such as about 7.5 mm to about 40 mm, about 10 mm to about 30 mm, about 12.5 mm to about 25 mm, about 15 mm to about 22.5 mm, or about 17.5 mm to about 20 mm, including all ranges and sub-values therebetween.

In some variations, the adapter may comprise one or more of a lumen, channel, and central aperture configured to at least partially surround and/or receive the end effector therethrough. A width or diameter of the lumen (i.e., an inner dimension of the adapter) may be about equal to or greater than a width or diameter of the end effector. For example, the adapter lumen may be configured to contact (e.g., hug, surround) at least a portion of (e.g., an entirety of) a perimeter of the portion of the end effector received through the lumen. In some variations, a width or diameter of the lumen may be about 1 mm to about 30 mm, such as about 2.5 mm to about 25 mm, about 5 mm to about 20 mm, about 7.5 mm to about 15 mm, or about 10 mm to about 12.5 mm, including all ranges and sub-values therebetween. In some variations, a width or diameter of the lumen may be about equal to or less than 30 mm, about equal to or less than 20 mm, about equal to or less than 15 mm, about equal to or less than 10 mm, or about equal to or less than 5 mm. In some variations, the lumen may have a constant or substantially constant width or diameter along a longitudinal axis of the adapter. In some variations, the lumen may have a varied (e.g., sloped or tapered) width or diameter therethrough. Moreover, a width or diameter of the adapter body (i.e., an outer dimension of the adapter) may be about equal to or less than a width or diameter of a recess of the clamp. In some variations, a width or diameter of the adapter body may be about 5 mm to about 50 mm, such as about 7.5 mm to about 40 mm, about 10 mm to about 30 mm, about 12.5 mm to about 25 mm, or about 15 mm to about 20 mm, including all ranges and sub-values therebetween. Further, the lumen of the adapter may generally comprise a shape that is complementary to (e.g., corresponds to) a shape of the end effector. In some variations, the adapter (and end effector) may comprise a cylindrical shape, or a circular cross-sectional shape. In some variations, the width or diameter of the lumen may be adjustable.

In some variations, the adapter may be fabricated from one or more materials, such as a plastic, metal, ceramic, polymer, and/or composite material. In some variations, the adapter may be manufactured using 3D printing, casting, injection molding, thermoforming, or any other suitable manufacturing process.

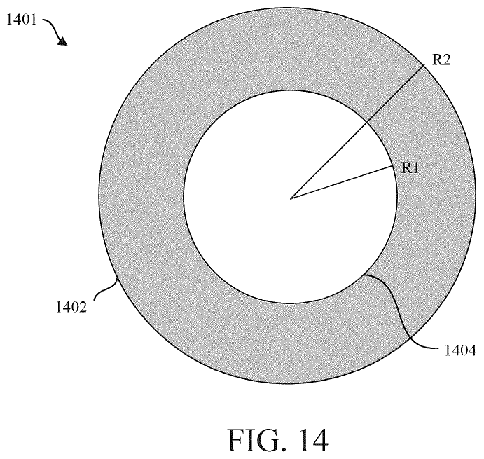

A cross-sectional view of an illustrative representation of an adapter 1401 for use with the systems herein is depicted in FIG. 14. As shown, the adapter 1401 may comprise a body 1402 and a lumen 1404 therethrough. The body 1402 and the lumen 1404 may comprise a complementary cross-sectional shape. The lumen 1404 may have a first dimension (e.g., radius or diameter) that is less than a second dimension (e.g., radius or diameter) of the body 1402. In some variations, a difference in length between the first and second dimensions may be about 0.01 mm to about 25 mm, such as about 0.1 mm to about 20 mm, about 0.25 mm to about 15 mm, about 0.5 mm to about 10 mmm, about 0.75 mm to about 9 mm, about 1 mm to about 8 mm, about 1.25 mm to about 7 mm, about 1.5 mm to about 6 mm, about 1.75 mm to about 5.5 mm, about 2 mm to about 5 mm, about 2.25 mm to about 4.5 mm, about 2.5 mm to about 4 mm, about 2.75 mm to about 3.5 mm, or about 3 mm to about 3.25 mm, including all ranges and sub-values therebetween.

In some variations, the adapter body may comprise folds or ribs configured to increase the strength and bending stiffness of the body without affecting the thickness of the body wall. This feature may enable the adapter to withstand a clamp force necessary to stabilize and secure the end effector within the clamp (via the adapter). Additionally, or alternatively, in some variations the adapter body may comprise a base at one or both of a first and second end of the body. The base may comprise a rim or planar surface (e.g., plate) that extends radially from the perimeter of the body (e.g., transversely to the longitudinal axis of the body). The base may be configured to abut a portion of the clamp so as to reduce or prevent movement of the adapter relative to the clamp and further stabilize a position of the end effector relative to the clamp. In some variations, an outer dimension (e.g., width or diameter) of the base may be about equal to or greater than an inner dimension of the recess(es) of the clamp to prevent the adapter from moving within the clamp.

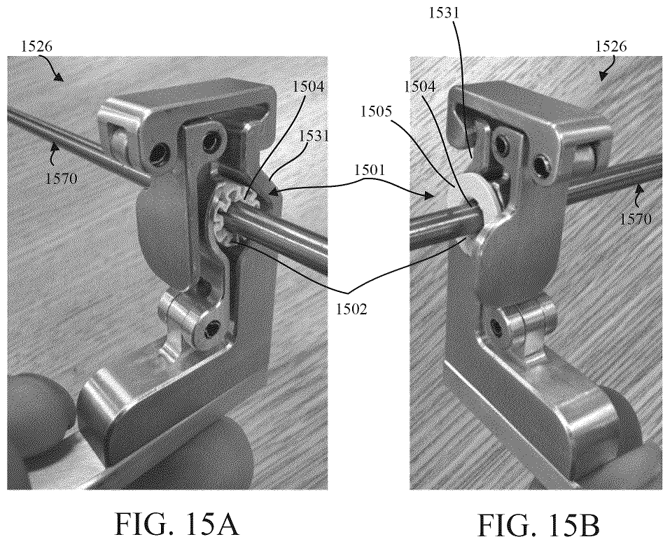

FIGS. 15A and 15B show an exemplary variation of an adapter 1501 within a clamp 1526 (e.g., an over-center clamp). The adapter 1501 may comprise a substantially cylindrical body 1502. The walls of the body 1502 may be folded. The adapter 1501 may comprise a central lumen 1504 extending through an entire length of the body 1502. A diameter of the lumen 1504 may be about equal to or greater than a diameter of an end effector 1570 so that the end effector 1570 may be fed through the lumen 1504. Further, the adapter 1501 may comprise a base 1505 that radially surrounds an opening of the lumen 1504 on a first side of the adapter 1501. The base 1505 may be configured to overlap with a wall defining a first recess 1531 of the clamp 1526 to prevent the adapter 1501, and thus the end effector 1570, from moving (e.g., sliding) within the clamp 1526 when it is in the closed (e.g., locked) position.

### 4. Support Arm

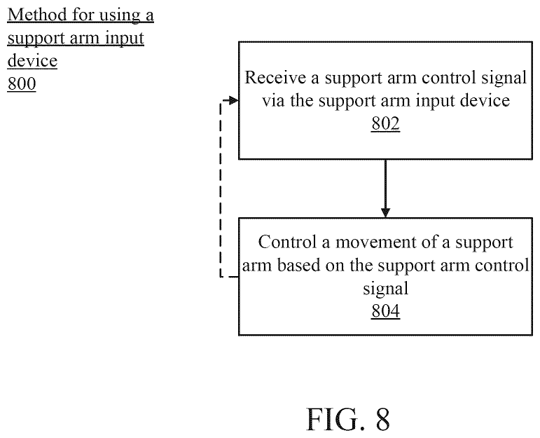

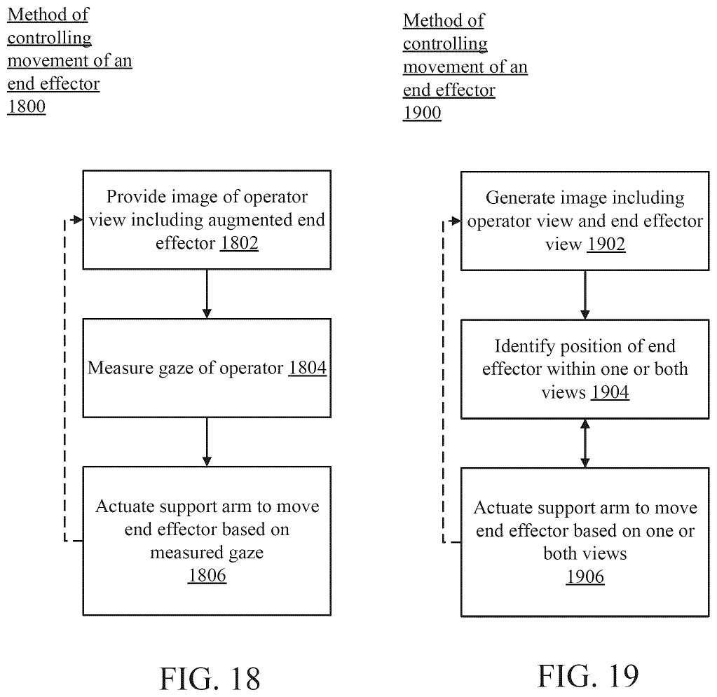

The surgical systems described herein may comprise one or more support arms (“robotic arms”) configured to releasably couple to an end effector via an end effector connector. In some variations, a support arm may be configured to control a movement of the end effector during a robotic surgery procedure. For example, as described in detail below with reference to input device 600 of FIGS. 6A-6G, method 800 of FIG. 8, method 1800 of FIG. 18, and method 1900 of FIG. 19, the support arms described herein may receive a control signal for controlling support arm (and, effectively, end effector), where the control signal may be generated based on movement or measurement of an operator.

The support arms herein may be configured to move over all areas of a patient body in up to three dimensions and may also maintain the end effector at an orientation perpendicular to a surface of the patient. The support arm may be configured to move in a plurality of degrees of freedom (e.g., three, four, five, six, seven, eight degrees of freedom). A support arm may comprise one or more motors configured to translate and/or rotate the joints and move the support arm to a desired location and orientation. In some variations, a position of the support arm may be temporarily locked to fix a position of the end effector (e.g., within a body cavity). The support arm may be mounted to any suitable object, such as a medical cart, furniture (e.g., a bed rail), a wall, a ceiling, or may be self-standing (e.g., on the ground). Additionally, or alternatively, the support arm may be configured to be moved manually by, for example, a single operator without the assistance of a second person. Once manually moved by the single operator, the support arm may be locked to the manually mounted position. The support arm may be configured to carry a payload comprising the support arm and one or more of the end effector connector, the adapter, the end effector, the sterile drape, and any tissue coupled to the end effector (e.g., an organ, such as a gallbladder, held by a grasper).

In some variations, the relative positions of a patient platform (or other surgical space), patient, operator, and support arm may be configured to aid the ergonomics of a surgical procedure. In some variations, an operator may be located on a first side of a patient platform during a surgical procedure. In some variations, a support arm may be mounted to a base. In such variations, the base may be located on the ground and along a second side of a patient platform adjacent the first side of the patient platform to maximize a range of the support arm. The first side of the patient platform may be perpendicular to the second side of the patient platform. For example, the support arm may extend from its base above and over (e.g., across) a patient disposed on the patient platform. The base may be located closer to a mid-point of the second side of the patient platform rather than an intersection of the first side and the second side in order to maximize the flexibility of the support arm to reach an access site of a patient. In some variations, a base of the support arm (e.g., robotic arm) may be coupled to a lateral side of a patient platform. In some variations, the base may be moveable. For example, the base may comprise wheels to facilitate moving the support arm, via the base, about a surgical space. This may provide flexibility to customize the ergonomics of a given surgical procedure.

### 4.1. Coupling Mechanism

The support arms herein may comprise a coupling mechanism for coupling end effector connectors to the support arms. In some variations, the coupling mechanism may be at a distal end of a support arm (e.g., coupling mechanism 260 in FIGS. 2A-2C). In some variations, the coupling mechanism may be coupled to the end effector connector with a sterile drape disposed therebetween. Referring to exemplary FIG. 2A, the annular portion 240 of the end effector connector 210 may be configured to couple to and decouple from the coupling mechanism 260 of the support arm. The coupling mechanism 260 may include a cylindrical housing 262 around which the annular portion 240 may surround when coupled to the coupling mechanism 260. That is, the annular portion 240 may be configured to fit around a circumference of the cylindrical housing 262. The cylindrical housing 262 may extend distally from the support arm. The cylindrical housing 262 may include an actuator 261 disposed within the cylindrical housing 262. The actuator 261 may be configured to be actuated by an operator to release the annular portion 240 of the end effector connector 210 from the cylindrical housing 262 of the coupling mechanism 260 (and thus from the support arm 280).

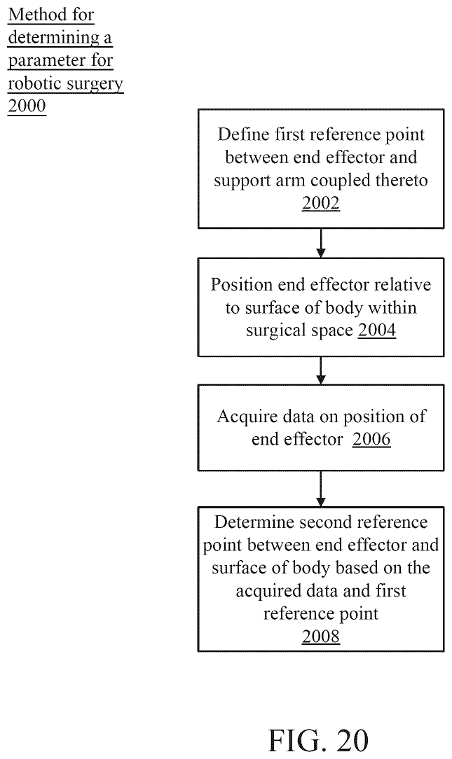

In some variations, the coupling mechanism 260 may include one or more indictors, such as indictor 266, for providing feedback (e.g., audio and/or visual feedback) to an operator regarding the status of the coupling between the end effector 270 and the support arm 280. For example, when the end effector 270 and the support arm 280 are coupled, the indicator 266 may visually indicate the coupling via a first colored light (e.g., a blue LED light). When the end effector 270 and the support arm 280 are not coupled, the indicator 266 may visually indicate the lack of coupling via a second, different colored light (e.g., a red LED light) or by no light. In some variations, the indicator 266 may comprise an actuator, such as a control button. In some variations, the actuator may be used to determine surgical parameters. For example, upon actuation (e.g., pushing) of the actuator, the system 200 may be configured to acquire data indicating a position of the support arm 280 and/or end effector 270 within the surgical space. That is, actuating the actuator may cause the system 200 (e.g., via a processor) to determine and store information modeling the position of the support arm 280 and/or end effector 270 within the surgical space. This data may be used to determine other parameters such as a surgical reference point (e.g., an incision on a body of a patient) and/or a dimension (e.g., length) of an end effector. This function is described in more detail with respect to method 2000 of FIG. 20.

In some variations, the coupling mechanism 260 may include switch 265 located on grip 263 for manually manipulating a position of the robotic surgery system 200 and locking the system 200 in a fixed position (e.g., a rotational and/or translational position). For example, an operator may actuate the switch 265 by applying continuous pressure to the switch 265 with a first hand (e.g., a finger of the first hand). During this period of actuation, the operator may simultaneously grip the grip 263 with the first hand and use the grip 263 to steer or otherwise reposition the robotic surgery system 200. When the operator removes pressure from the switch 265, the robotic surgery system 200 may be locked in place. While FIGS. 2A-2B depict the switch 265 on a top wall of the grip 263, it should be understood that the switch 265 may also be situated on a side or bottom wall of the grip 263.

With the end effector suspended or held at a desired location by the support arm, an operator and/or controller may move at least a portion of the end effector externally of a patient. The support arm may be, for example, an articulated robotic arm, SCARA robotic arm, and/or linear robotic arm. The support arm may comprise one or more segments coupled together by a joint (e.g., shoulder, elbow, wrist) configured to provide a single degree of freedom. Joints are mechanisms that provide a single translational or rotational degrees of freedom. For example, the support arm may have six or more degrees of freedom. The set of Cartesian degrees of freedom may be represented by three translational (position) variables (e.g., surge, heave, sway) and by the three rotational (orientation) variables (e.g., roll, pitch, yaw). In some variations, the support arm may have less than six degrees of freedom.

As described above with respect to FIGS. 2A-2I, an end effector connector (e.g., end effector connector 220) may comprise a housing (e.g., housing 220), an arm (e.g., arm 230), and a housing release mechanism (e.g., housing release mechanism 250) therebetween configured to releasable couple the housing to the arm. The end effector connector may be configured to removably couple the end effector to a support arm. For example, the end effector connector may include an annular portion (e.g., annular portion 240) configured to releasably couple the arm (with or without the removably attached housing and end effector) to the support arm (e.g., support arm 280). In particular, the annular portion may be configured to couple to and decouple from the coupling mechanism (e.g., coupling mechanism 260) at a distal end of the support arm. The coupling mechanism may allow an operator to couple and/or decouple the end effector connector to/from the support arm using a single hand of an operator. For example, the coupling mechanism may provide a pneumatic coupling, interference-fit coupling, and/or snap-fit coupling of the end effector connector to the support arm.

Exemplary variations of the coupling between the end effector connector and the coupling mechanism of the support arm are provided in FIGS. 3A-3D, which depict perspective views of the robotic surgery system 300. FIG. 3E depicts a perspective view of an illustrative variation of the end effector connector 310 of the robotic surgery system 300. FIGS. 3F and 3G depict side views of the coupling mechanism 320 of the robotic surgery system 300, and FIG. 3H depicts a perspective view of the coupling mechanism 320 of the robotic surgery system 300. The end effector connector 310 may be coupled to the arm 314 at a first (i.e., distal) end and may be releasably coupled to the coupling mechanism 320 at a second (i.e., proximal) end. When the end effector connector 310 is coupled to the coupling mechanism 320, as depicted in exemplary FIG. 3A, the annular portion 312 of the end effector connector 310 may be oriented around and along the cylindrical housing 324 of the coupling mechanism 320. For example, an interior sidewall 311 of the annular portion 312 may surround and contact at least a portion of the circumference of the cylindrical housing 324 (e.g., a distal portion of the cylindrical housing 324).

The coupling mechanism 320 may include the sleeve 322 disposed around a portion of the circumference of the cylindrical housing 324 and translatable along a length of the cylindrical housing 324. In particular, the sleeve 322 may be biased toward a distal end of the cylindrical housing 324 via pressure applied by the springs 323. That is, absent an opposite, resistance force to maintain the sleeve 322 at or adjacent a proximal end of the cylindrical housing 324, the sleeve 322 may be forced toward the distal end of the cylindrical housing 324, preventing other components (e.g., the annular portion 312) from simultaneously maintaining a position around the distal end of the cylindrical housing 324.

Accordingly, to couple the annular portion 312 to the cylindrical housing 324 (i.e., maintain a position of the annular portion 312 around the distal end of the cylindrical housing 324), the coupling mechanism 320 may include projections 326 for holding the annular portion 312 around the cylindrical housing 324 and against a distal end of the sleeve 322. Thus, the annular portion 312 may apply a resistant force to the pressurized sleeve 322, preventing the sleeve 322 from translating distally along the cylindrical housing 324 and ejecting the annular portion 312 therefrom. The projections 326 may be configured to translate laterally through a sidewall of the cylindrical housing 324. For example, the projections 326 may be configured to at least partially extend beyond the exterior of the cylindrical housing 324 to align with and contact the interior sidewall 311 of the annular portion 312.

In some variations, the interior sidewall 311 of the annular portion 312 may include one or more grooves configured to receive one or more complementary projections 326 when the annular portion 312 is appropriately aligned with the sleeve 322. The geometry of the grooves may be opposite to the geometry of the projections 326. For example, the projections 326 may be convex relative to the interior sidewall 311 of the annular portion 312, and the grooves within the interior sidewall 311 may be concave and sized to at least partially receive the projections 326 therein. Proper rotational alignment of the annular portion 312 and the sleeve 322 may be achieved by aligning sleeve protrusions 323 and indentations 325 with complementary annular portion indentations 313 and protrusions 315 of the annular portion 312 of the end effector connector 310. For example, a distal end of the sleeve 322 may include one or more plateau-shaped protrusions and indentations configured to align with one or more complementary plateau-shaped indentations and protrusions of a proximal end of the annular portion 312. Each of a plurality of sleeve protrusions 323 and indentations 325 and a plurality of complementary annular portion indentations 313 and protrusions 315 may comprise a same or different shape. For example, FIG. 3E depicts the annular portion 312 having a plurality of indentations 313, each having a unique length and width. Correspondingly, the sleeve 322 may have a plurality of protrusions 323, each having a length and width corresponding to one of the plurality of indentations 313. As such, the annular portion 312 may only be configured to align with the sleeve 322 in a single rotational alignment. This feature may minimize operator error when coupling the annular portion 312 to the coupling mechanism 320 by ensuring that the components are successfully coupled using the single functional orientation.

Additionally, the projections 326 may be configured to retract at least partially within an interior of the cylindrical housing 324 upon actuation to release the end effector 310 from the coupling mechanism 320. The actuation may be induced via the actuator 321. The actuator 321 may be housing within the cylindrical housing 324 and may be translatable within the cylindrical housing 321. For example, the actuator 321 may be configured to receive an upward (e.g., proximal) force by an operator that causes the actuator 321 to translate proximally within the cylindrical housing 324 from a distal-most end of the cylindrical housing 324. As described below with respect to FIGS. 4A-4C, the actuator 321 may be a spring-loaded component. In some variations, the actuator 321 may include grooves (not shown) configured to allow the projections 326 to at least partially retract therein. Accordingly, when the grooves of the actuator 321 are aligned with the projections 326 (e.g., via translation of the actuator 321 within the cylindrical housing 324), the projections 326 may at least partially retract within the actuator grooves, removing the force applied to the annular portion 312 by the projections 326, and allowing the pressurized sleeve 322 to translate distally and eject the annular portion 312 from the cylindrical housing 324. Like the grooves of the interior sidewall 311 of the annular portion 312, the geometry of grooves of the actuator may be complementary to the geometry of the projections 326. In some variations, the projections may have a constant geometry (e.g., they may be spherical), and the actuator grooves and annular portion grooves may have a same or substantially similar geometry.

In some variations, the coupling mechanism 320 may include one or more minor indictors, such as minor indictors 360, and/or one or more major indicators, such as major indicator 370, for providing feedback (e.g., audio and/or visual feedback) to an operator regarding the status of the coupling between the annular portion 312 and the coupling mechanism 320. For example, when the annular portion 312 and the coupling mechanism 320 are coupled, the minor indicators 360 and/or the major indicator 370 may visually indicate the coupling via a first colored light (e.g., a blue LED light). When the annular portion 312 and the coupling mechanism 320 are not coupled, the minor indicators 360 and/or the major indicator 370 may visually indicate the lack of coupling via a second, different colored light (e.g., a red LED light) or by no light.

FIGS. 4A-4C depict cross-sectional views of the robotic surgery system 400. In particular, these figures depict how actuation (e.g., distal translation) of the actuator 421 causes the annular portion 412 of the end effector connector 410 to release from the coupling mechanism 420.

FIG. 4A shows a first configuration of the coupling mechanism 420 when it is coupled to the end effector connector 410. In this configuration, end effector connector 410 and the sleeve 422 may be rotationally aligned, allowing the projections 426 to be at least partially received within complementary grooves 419 of the annular portion 412. Additionally, the actuator 421 may be biased toward a distal end of the cylindrical housing 424 via the springs 427.

The second configuration of the coupling mechanism 420 shown in FIG. 4B may occur upon actuation (e.g., via a hand of an operator) of the actuator 421. As depicted, actuation of the actuator 421 may correspond to proximal translation of the actuator 421 through the cylindrical housing 324. The actuator 421 may be configured to be translated until the actuator grooves 429 are laterally aligned with the projections 426. Thus, the projections 426 may shift inward through the cylindrical housing 424 and toward the actuator grooves 429.

The lateral shift of the projections 426 at least partially within the actuator grooves 429 may allow the coupling mechanism 420 to transition to a third configuration, such as that depicted in FIG. 4C. Specifically, the projections 426 may no longer be preventing the pressurized sleeve 422 (e.g., via springs 423) from ejecting the annular portion 412 from the cylindrical housing 424. Accordingly, the sleeve 422 may translate distally along the cylindrical housing 424, pushing the annular portion 412 proximally along the cylindrical housing 424, and ultimately pushing the annular portion 412 off of the cylindrical housing 424.

### 5. Sterile Covering

The surgery systems described herein may include one or more sterile coverings (e.g., sterile drape, sterile bag) configured to create a sterile barrier around portions of the surgery system. In some variations, the surgery system may include one or more sterile coverings to form a sterile field. For example, as shown in robotic surgery system 500 of FIGS. 5A-5D, a sterile covering 520 may be placed between the support arm 580 and the patient (not shown) or surgical space, forming a barrier between a sterile side and a non-sterile side. The sterile side may include the patient (not shown) end effector (not shown), end effector connector 510, and operator, and the non-sterile side may include the support arm 580 and the coupling mechanism 560 coupled to a distal end of the support arm 580. In some variations, the sterile covering 520 may be disposed between the annular portion 512 of the end effector connector 510 and the sleeve 562 of the coupling mechanism 560 such that a first side (e.g., exterior) of the sterile covering faces the end effector connector 510 and a second side (e.g., interior) of the sterile covering faces the support arm 580.

Additionally, or alternatively, one or more components of the system may be sterilizable. The sterile covering may, for example, be a sterile drape configured to cover at least a portion of a system component described herein.

For example, the sterile covering may be configured to create a sterile barrier with respect to a support arm. In some variations, the sterile bag may be clear and allow an operator to visualize and manually manipulate a position of the end effector by, for example, an operator grabbing a handle of a support arm or a handle attached to the end effector through the sterile bag. The sterile covering may conform tightly around one or more system components or may drape loosely so as to allow components to be adjusted within the sterile field (e.g., attachment and release of an end effector from a support arm via an end effector connector).

### 6. Controller

The systems herein may comprise a controller (e.g., one or more controllers, a plurality thereof) configured to control operation of the support arm(s) in preparation for and/or during a surgical procedure. The controllers herein may be configured for one or more of receiving surgical parameters and/or control signals (e.g., via an input device), processing control signals (e.g., via a processor), determining information related to a surgical procedure (e.g., system parameters), storing information related to a surgical procedure (e.g., via a memory), providing information related to a surgical procedure (e.g., via an output device), and communicating with other controllers (e.g., via a communication device). Accordingly, as shown in FIG. 1, a controller 120 may comprise (e.g., be operably coupled with) one or more of an input device 122, a processor 124, a memory 126, a communication device 128, and an output device 130. Each of these components will be described in further detail below.

### 6.1. Input Device

The input devices herein may be configured to control movement of a support arm (and an end effector coupled thereto) by receiving and generating a control signal indicative of such movement. For example, an input device may be configured to receive, via a physical or electrical force or signal, operator input to control a support arm, and may be configured to transmit the input to the support arm (e.g., a processor thereof) to actuate movement of the support arm. In some variations, the support arm movement may include a series of movements controlled by a series of corresponding control signals, resulting in motion of the support arm. Motion of the support arm may be defined by at least three degrees of freedom, such as three, four, five, or six degrees of freedom.

In some variations, each of a plurality of support arm control signals may be generated (via the input device) and/or transmitted (e.g., to a support arm or end effector) independently. That is, in some variations, only one support arm control signal may be transmitted to a robotic system component from the input device at a time. The control signals may be processed (e.g., by a support arm processor) independently, multiple control signals may be processed together, or a combination of these processing methods may occur.

Additionally, or alternatively, the input devices herein may comprise a device for providing a user interface configured to receive information related to a patient, surgical procedure, and/or components of the robotic surgery system. The device may be, for example, a display. Information received by a user interface may be processed in order to plan (e.g., determine and set parameters for) a surgical procedure. In some variations, the user interface may be configured to provide one or more (e.g., a series of) prompts for an operator to determine one or more parameters for the surgical procedure.

In some variations, the systems herein may comprise a plurality of input devices, such as two or more or at least two input devices. In some variations, a plurality of input devices may comprise one or more types of input devices. For example, as described below, the surgery systems herein may comprise one or more of a foot-actuated input device, a gaze-actuated input device, and a user interface. In some variations, an input device may include an AR or VR tool configured to enhance visualization of a surgical procedure. In some variations, two or more of a plurality of input devices may be communicably coupled via a network (e.g., wireless or wired network).

Referring again to FIG. 1, the input device 122 of a robotic surgery system 100 may serve as a communication interface between an operator and the surgery system 100. The input device 122 may be configured to receive one or both of input data and output data from one or more of an operator (e.g., a foot or gaze of the operator), the support arm 112, the sensor 119, the end effector 118, and the output device 130. For example, operator control of an input device 122 (e.g., foot controller, AR or VR device, joystick, keyboard, display, touch screen) may be processed by processor 124 and memory 126 to output an interpretable control signal to one or more support arms 120 and/or effectors 118. As another example, images generated by an end effector 118 comprising a visualization device (e.g., an endoscope) may be received by input device 122, processed by processor 124 and memory 126, and displayed by the output device 130 (e.g., a monitor display). As yet another example, the input device 122 may be configured to capture an image (e.g., a live image of an operator's field of view) and output the image via the output device 130. Additionally, or alternatively, sensor data from the sensor(s) 119 may be received by the input device 122.

In some variations, a single operator may control one or more components of a surgery system 100 using one or more input devices 122. Each of a plurality of input devices may be configured to control one or more support arms 120 and/or end effectors 118. In some variations, the input device 122 may be configured to switch operator control from a first support arm to a second support arm. In some variations, the input device 122 may include at least one switch or actuator (e.g., a virtual actuator) configured to generate a control signal.

The input device 122 may be coupled to a support arm 120 and/or disposed on a patient platform or medical cart adjacent to the patient and/or operator. For example, the input device 122 may comprise a foot-actuated device configured to be adjustably positioned on a floor of a surgical space. As another example, the input device 122, which may be an AR or VR device, may include a headset, goggles, glasses, or contact lens(es) configured to be worn by an operator. Alternatively, the input device 122 may be mounted to any suitable object, such as furniture (e.g., a bed rail), a wall, a ceiling, or may be self-standing.

The input device 122 may be configured to receive a control signal from an operator. Nonlimiting examples of the control signal may include an applied force, a measurement of an operator parameter (e.g., operator gaze), a movement signal, a device switch signal, an activation signal, and/or a magnetic field strength signal. In some variations, the control signal may include one or more control signals, such as at least two control signals, or a plurality of control signals. The input device 122 may be configured to transmit and/or receive signals to and/or from other components of the robotic surgery system 100 via a wired and/or wireless connection. For example, the input device may comprise a wired and/or wireless transmitter configured to transmit a control signal to a wired and/or wireless receiver of a controller (e.g., via the communication device 128). A movement control signal (e.g., for the control of movement, position, and/or orientation of a support arm or end effector) may control movement in one, two, three, four, five, or six degrees of freedom (i.e., up/down, forward/back, left/right, pitch, roll, and/or yaw).

### Foot-Actuated Input Device

In some variations, the input device may comprise a foot-actuated input device (“foot controller”) configured to receive input from a foot of an operator. In some variations, a foot-actuated input device may be configured to operate one or more support arms and end effectors of a robotic surgery system described herein. Here, the support arm control signal may include one or more support arm switch commands corresponding to a toe or front foot (“forefoot”) movement of the single foot. Additionally, or alternatively, in some variations, the support arm control signal may include one or more support arm switch commands corresponding to a heel (“hindfoot”) movement of the single foot. In some variations, the support arm control signal may be generated by a translation motion of the operator (e.g., a foot of the operator) that corresponds to a translation motion (e.g., in the X, Y, or Z directions) of the support arm. In some variations, the support arm control signal may be generated by a rotational motion of the operator (e.g., a foot of the operator) that corresponds to a rotational motion (e.g., in roll, pitch, or yaw) of the support arm. For example, the support arm control signal may comprise a downward motion of the support arm corresponding to a rotation in pitch of a single foot of an operator. As another example, the support arm control signal may comprise a lateral motion of the support arm corresponding to a rotation in yaw of the single foot of the operator. Such rotation may be achieved by, for example, a flexion motion of the foot.

FIGS. 6A-6G depict an exemplary input control scheme for generating a support arm control signal corresponding to various movements of a single foot of an operator. The foot movements of the operator may be used actuate switches of the input device that are configured to control (e.g., adjust, maintain, initiate, and/or arrest) a movement of one or more support arms configured to receive the control signal from the input device. FIG. 6A depicts a perspective view of input device 600 of a robotic surgery system. FIGS. 6B-6D depict sectional top views of input device 600 of a robotic surgery system. FIGS. 6E and 6F depict a front view and a sectional front view, respectively, of input device 600 of a robotic surgery system. FIG. 6G depicts a back view of input device 600 of a robotic surgery system.

The input device 600 may include a base 640 having a midfoot portion 642 (e.g., a midfoot rest portion) set of actuators coupled thereto. The set of actuators may include a set of forefoot actuators 610 (“first forefoot actuator” or “first actuator”), 620 (“second forefoot actuator” or “second actuator”), 614 (“third forefoot actuator” or “fourth actuator”), and a hindfoot actuator 630 (“third actuator”). The first forefoot actuator 610 may be configured to control rotational movement (e.g., one or more of roll, pitch, and yaw rotation) of a support arm and/or end effector. The second forefoot actuator 620 may be configured to control translational movement (e.g., distal translational movement relative to an operator) of the support arm and/or end effector. The hindfoot actuator 630 may also be configured to control translational movement (e.g., proximal translational movement relative to an operator) of the support arm and/or end effector. The third forefoot actuator 614 may be configured to transfer transmissions of the first, second, and fourth support arm control signals from a first support arm to a second support arm. For example, the fourth support arm control signal may transfer operator control from the first support arm to the second support arm by generating a power off signal for turning the first support arm off, and by generating a power on signal for turning the second support arm on.

One or more actuator of the set of actuators 610, 614, 620, 630 may be pressure sensitive. For example, each of the actuators 610, 614, and 620 may be configured to be actuated with contact by a forefoot (e.g., one or more toes or a proximal foot portion) of the operator. Similarly, the hindfoot actuator may be configured to be actuated with contact by a hindfoot (e.g., a heel or a distal foot portion) of the operator.

In some variations, motion of an operator foot may correspond to motion of a support arm. In some variations, a forward motion (e.g., translation) of the foot may activate the second forefoot actuator 620 and correspond to a forward motion (e.g., distal translation relative to the operator) of the support arm and any end effector coupled thereto. In some variations, a backward motion (e.g., translation) of the foot may activate the hindfoot actuator 630 and may correspond to a backward motion (e.g., proximal translation relative to the operator) of the support arm and any end effector coupled thereto. In some variations, a downward motion of the forefoot (e.g., extension) may activate the first forefoot actuator 610 and may correspond to a downward rotation (e.g., pitch rotation) and any end effector coupled thereto In some variations, an upward motion of the forefoot (e.g., flexion) may activate the first forefoot actuator 610 and may correspond to an upward rotation (e.g., pitch rotation) of the support arm and any end effector coupled thereto. In some variations, a lateral motion of the forefoot may activate the first forefoot actuator 610 and may correspond to a lateral rotation (e.g., yaw rotation) of the support arm and any end effector coupled thereto.

In some variations, a downward motion of the forefoot may activate the third forefoot actuator 614 and may correspond to a device switching signal. For example, activating the third forefoot actuator 614 may switch input device 600 control between a first support arm, a second support arm, an end effector, and the like. In some variations, the third forefoot actuator 614 may be coupled to an exterior top surface of the first forefoot housing 612. The third forefoot actuator may be configured to be activated by an underfoot of the operator. As such, a downward motion of the forefoot to activate the third forefoot actuator 614 may necessarily be preceded by an upward repositioning motion of the entire foot relative to the input device 600. This preceding motion may reduce accidental activation of the third forefoot actuator 614.

In some variations, one or more of the actuators 610, 614, 620, 630 may be translatable along the base 640. For example, the second forefoot actuator 620 may be include a track (not shown) for providing a limited range of translational movement for the second forefoot actuator 620, where the second forefoot actuator 620 may be configured to receive a forward (i.e., distal) foot movement resulting in translation of the second forefoot actuator 620 along the lateral portion 644. Similarly, the hindfoot actuator 630 may be include a track (not shown) for providing track providing a limited range of translational movement for the hindfoot actuator 630, where hindfoot actuator 630 may be configured to receive a backward (i.e., proximal) foot movement resulting in translation of the hindfoot actuator 630 along the distal end of the midfoot portion 642. Additionally, or alternatively, one or both of the top surfaces of the second forefoot actuator 620 and the hindfoot actuator 630 may be configured to receive an underfoot of the operator to generate the corresponding support arm and/or end effector movement control signal.

The operator may stand on the input device 600 such that, at a resting position, none of the forefoot, the midfoot, and the hindfoot may activate (e.g., contact) any of the actuators 610, 614, 620, 630. In some variations, the operator may operate the input device 600 from a sitting position. Additionally, or alternatively, operator actuation of the actuators 610, 614, 620, 630 may be limited to independent actuation of a single actuator 610, 614, 620, 630. A geometry of the base 540 may allow for the operator to rest the controlling foot on the midfoot portion 642 and/or may prevent simultaneous actuation of two or more actuators 610, 614, 620, 630. For example, the base 640 may be a low-profile base having a thickness of about 0.1 cm to about 5 cm (e.g., about 0.25 cm to about 4 cm, about 0.5 cm to about 3 cm, about 0.75 cm to about 2 cm, or about 1 cm to about 1.5 cm). The small thickness of the base 640 may allow an operator to easily step and out of the midfoot portion 642. Additionally, the actuators 610, 614, 620, 630 may be coupled to separate portions of the base 640 to prevent simultaneous actuation of two or more actuators 610, 614, 620, 630. For example, the first forefoot actuator 610 and third forefoot actuator 614 may be coupled to a distal portion 641 of the base 640, the hindfoot actuator 630 may be coupled to a proximal portion 643 of the base 640, and the second forefoot actuator 620 may be coupled to a lateral portion 644 (e.g., a proximal portion of the lateral portion 644) of the base 640. The distal and proximal portions 641, 643 of the base 640 may be aligned along a shared longitudinal axis of the input device 600. The lateral portion 644 may have a central longitudinal axis that is obliquely angled relative to the shared longitudinal axis of the distal and proximal portions 641, 643, resulting in an asymmetrical geometry of the input device 600.

Moreover, the input device 600 may include housings 612 (“first forefoot housing”), 622 (“second forefoot housing”), and 632 (“hindfoot housing”) to guide and/or limit movement of one or both of the foot of the operator. For example, one or more of the housings 612, 622, 632 may include at least one wall extending vertically from the base 640. The wall may border a corresponding actuator (610, 614, 620, 630) such that the foot of the operator may not move beyond the wall of the housing (612, 622, 632). Thus, a single foot of an operator may be configured to control the input device 600 in a robotic surgery system while leaving the operator's visual attention and hands available for other tasks.

In some variations, one or more of the actuators 610, 614, 620, 630 may include one or more switches configured to be actuated by the foot of the operator. For example, the first forefoot actuator 610 may include one or more switches 616 that are actuatable via manipulation of the forefoot receptacle 650 within the first forefoot housing 612. For example, the switches 616 may be coupled to an interior wall of the first forefoot housing 612 and the forefoot receptacle 650 may be releasably couplable to the interior wall of the first forefoot housing 612 via at least one magnet 618. Thus, the forefoot receptacle 650 may be suspended above a top surface of the base 640 and along the interior wall of the first forefoot housing 612. Accordingly, the forefoot receptacle 650 may receive rotational forefoot movement (e.g., forefoot rotation in one or more of roll, pitch, and yaw) to actuate the switches 616 and ultimately control a corresponding rotational movement of a support arm and/or end effector.

In some variations, the hindfoot actuator 630 may include an adjustment mechanism 636 for adjusting a position of the hindfoot actuator 630 relative to the hindfoot housing 632. For example, the hindfoot actuator 630 may be disposed upon a track allowing the hindfoot actuator 630 to be translatable along the base 640. The adjustment mechanism 636 may allow for a size (i.e., length) of the midfoot portion 642 to be adjustable, thereby allowing for the input device 600 accommodate a range of operator foot sizes. In some variations, the adjustment mechanism 636 may be a releasable lock that configured to maintain a desired position of the hindfoot actuator 630 relative to the hindfoot housing 632. In some variations, the desired position may include be first position of a plurality (e.g., a range) of positions of the hindfoot actuator 630 such that the hindfoot actuator 630 may be translatable along the base 640. That is, the first position may be an upper (i.e., distal-most) or lower (proximal-most) threshold for translational movement of the hindfoot actuator 630 within a range of available translational movement. In some variations, the adjustment mechanism 636 may have an at-rest configuration that locks the position of the hindfoot actuator 630. This at-rest configuration may prevent an operator from accidentally translating the hindfoot actuator 630 to a different position upon contact to the hindfoot actuator 630 with the foot of the operator.

In some variations, the input device 600 may include one or more additional switches that correspond to additional movements of a support arm (e.g., combination movements such as proximal or distal translation and rotation).

In some variations, the set of actuators 610, 614, 620, 630 may be programmed with different functions according to operator preference. In some variations, the set of actuators 610, 614, 620, 630 may include one or more of a mechanical switch, optical sensor, accelerometer (e.g., 3-axis), gyroscope (e.g., 3-axis), motion sensor, pressure sensor, magnetic sensor, combinations thereof, and the like. In some variations, the input device 600 may be configured to releasably couple to the foot of the operator foot may be releasably coupled (e.g., strapped) to the input device 600.

### Gaze-Actuated Input Device

In some variations, the input device may comprise a gaze-actuated input device (“gaze controller”) configured to receive input from one or both eyes of an operator. In some variations, a gaze-actuated input device may be configured to operate one or more support arms and end effectors of a robotic surgery system described herein. Here, the support arm control signal may be generated by measuring a gaze of the operator. For example, the gaze-actuated input device may comprise one or more (e.g., a plurality of) actuators each configured to respond to a gaze of the operator to generate a corresponding support arm control signal. In some variations, the plurality of actuators may be virtual actuators provided with an AR or VR tool configured to be worn over one or more eyes of an operator, such as a headset, goggles, glasses, contacts, and/or the like. The operator may actuate each virtual actuator by directing a gaze toward the actuator for a time period. Put another way, once a length of the gaze of the operator is determined to be about equal to or greater than a time period, the actuator at which the gaze is directed may transmit corresponding control signal to a support arm or end effector. In some variations, the time period may be about 0.1 seconds(s) to about 15 s, such as about 0.5 s to about 12.5 s, about 1 s to about 10 s, about 1.25 s to about 7.5 s, about 1.5 s to about 5 s, or about 1.75 s to about 2.5 s (including all ranges and subranges therebetween). In some variations, the time period may be adjusted (e.g., individually for each operator).

The gaze-actuated input device may comprise any suitable number of actuators (e.g., virtual actuators) to control movement of a support arm or end effector in one or more degrees of freedom, such as in one, two, three, four, five, or six degrees of freedom. For example, a gaze-actuated input device may comprise one or more of: a first actuator configured to translate a support arm along a first axis, a second actuator configured to translate the support arm in along a second, different axis, and a third actuator configured to translate the support arm along a third, different axis. Each of the first, second, and third axes may be one of the X, Y, or Z axes. In some variations, the gaze-actuated input device may comprise two separate actuators for translating the support arm in opposite directions along a same axis. Additionally, or alternatively, the gaze-actuated input device may comprise one or more of: a first actuator configured to rotate the support arm around a first axis, a second actuator configured to rotate the support arm around a second, different axis, and a third actuator configured to translate the support arm around a third, different axis. Accordingly, each one of these actuators may control pitch, yaw, or roll of the support arm. In some variations, the gaze-actuated input device may comprise two separate actuators for rotating the support arm in opposite directions around a same axis.

In some variations, the gaze-actuated input device may be activated and deactivated by a separate control signal. For example, another controller, such as a foot-actuated input device, may be configured to actuate an actuator on the gaze-actuated input device to activate the gaze-actuated input device. Such an actuator may be power control. Accordingly, in some variations, the gaze-actuated input device may be operably coupled to another input device of the robotic surgery system (e.g., via a network). Furthermore, the gaze-actuated input device may be coupled to an output device, such as a display (as described below) in order to provide actuators. For example, the gaze-actuated input device may comprise a combination input/output device configured to provide an augmented or virtual experience for an operator throughout a surgical procedure.

### User Interface

In some variations, an input device may include a device configured to generate a user interface to receive information related to a patient, surgical procedure, and/or components of the robotic surgery system. For example, the user interface may be configured to receive known dimensions (e.g., length, width or diameter, height) of an end effector to be used during a procedure so that the system (e.g., via a processor) can determine a position of a reference point between the end effector a body or object within the surgical space. Additionally, or alternatively, the user interface may be configured to receive an indication that one or more dimensions (e.g., length, width or diameter, height) of the end effector are unknown so that the system can determine the unknown dimension(s) via a triangulation procedure, as will be described in detail herein.

The user interface may be provided on a display, such as a display of a computer monitor, laptop, tablet, or other suitable mobile device.

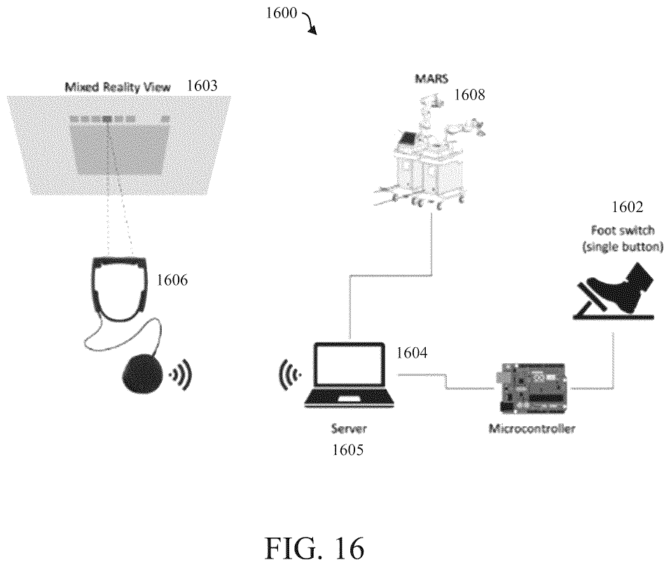

An exemplary configuration of a robotic surgery control system 1600 including a plurality of input devices is provided in FIG. 16. As shown, such a system may include a first input device 1602, which may be a foot-actuated device, a second input device 1604, which may be a display 1604 that provides a user interface, and a third input device 1606, which may comprise an AR or VR tool (e.g., a headset). One or both of the second input device 1604 and third input device 1606 may also function as an output device. For example, the second input device 1604 may be configured to provide feedback or medical information for an operator via the user interface, and the third input device 1606 may be configured to provide a mixed-reality image 1603 as an aid for the operator during a procedure. In some variations, the first input device 1602 may comprise a single-switch input device configured to activate and deactivate the third input device 1606 via movement of an operator foot. In some variations, the first input device 1602 may comprise a multi-switch input device configured to generate control signals corresponding to movement of the support arm(s) 1608. The support arm(s) 1608 may be actuated via one or both of the first, second, and third input devices 1602, 1604, 1606. Further, the first, second, and third input devices 1602, 1604, 1606 and the support arm(s) 1608 may be communicably coupled via a network server 1605, which may be wireless.

### 6.2. Processor

The controllers herein may include a processor. In some variations, the processor may be configured to operate a support arm (e.g., based on a control signal from an input device or controller). Additionally, or alternatively, the processor may be configured to process an image and transmit the processed image to an output device for providing to an operator. Additionally, or alternatively, a processor may be configured to execute a protocol for a surgical procedure according to a set of parameters received at or determined by the processor.

FIG. 1 illustrates a block diagram of a surgery system 100 including the processor 124, which may be in communication with one or more support arms 112 and/or end effectors 118. The processor 124 may be connected to the support arms 112 and/or end effectors 118 by wired or wireless communication channels. The processor 124 may be located in the same or different room as the patient. In some variations, the processor 124 may be coupled to a patient platform or disposed on a medical cart adjacent to the patient and/or operator. The processor 124 may be configured to control one or more components of the system 100, such as an end effector 118 that may visualize a body cavity or lumen, grasp tissue, retract tissue, hold and/or drive a needle, and the like. In some variations, the processor 124 may be configured to coordinate movement and orientation of end effectors 118 within a surgical space, body cavity, or lumen through corresponding movement and control of the support arm 112.

The processor 124 may be implemented consistent with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers or server computing devices such as routing/connectivity components, portable (e.g., hand-held) or laptop devices, multiprocessor systems, microprocessor-based systems, and distributed computing networks.

Examples of portable computing devices include smartphones, personal digital assistants (PDAs), cell phones, tablet PCs, phablets (personal computing devices that are larger than a smartphone, but smaller than a tablet), wearable computers taking the form of smartwatches, portable music devices, and the like, and portable or wearable augmented reality devices that interface with an operator's environment through sensors and may use head-mounted displays for visualization, eye gaze tracking, and user input.

The processor 124 may be any suitable processing device configured to run and/or execute a set of instructions or code and may comprise one or more data processors, image processors, graphics processing units, physics processing units, digital signal processors, and/or central processing units. The processor 124 may be, for example, a general purpose processor, a Field Programmable Gate Array (FPGA), an Application Specific Integrated Circuit (ASIC), configured to execute application processes and/or other modules, processes, and/or functions associated with the system and/or a network associated therewith. The underlying device technologies may be provided in a variety of component types such as metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, combinations thereof, and the like.

The systems, devices, and/or methods described herein may be performed by software (executed on hardware), hardware, or a combination thereof. Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the processor 124 may incorporate data received from memory 126 and operator input (e.g., via a user interface) to control one or more support arms 112 and/or end effectors 118. In some variations, images captured and/or received by the input device 122 may undergo processing by the processor 124 and/or may be stored by the memory 126. For example, an image captured or received by the input device 122 may be processed to enhance the image or to overlay graphics and/or reference markers onto the image. In some variations, two or more images may be combined (e.g., a first image may be overlayed onto a second image) by the processor 124. Accordingly, in some variations, the processor 124 may be utilized to provide an augmented or virtual reality experience for an operator during a surgical procedure.

### 6.3. Memory

The memory 126 may store instructions to cause the processor 124 to execute modules, processes, and/or functions associated with the system 100. Some variations of the memory 126 described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as air or a cable). The media and computer code (also may be referred to as code or algorithm) may be those designed and constructed for a specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical discs; solid state storage devices such as a solid state drive (SSD) and a solid state hybrid drive (SSHD); carrier wave signal processing modules; and hardware devices that are specially configured to store and execute program code such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

### 6.4. Communication Device

In some variations, controllers 120 described herein may communicate with networks and computer systems through a communication device 128. In some variations, a controller 120 may be in communication with other devices (e.g., controllers, systems) via one or more wired and/or wireless networks. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication. In some variations, the network interface 116 may comprise a radiofrequency receiver, transmitter, and/or optical (e.g., infrared) receiver and transmitter. The communication device 128 may communicate by wires and/or wirelessly with one or more of the support arm 112, end effector 118, sensor 119, input device 122, output device 130, network, database, server, combinations thereof, and the like.

### 6.5. Output Device

In some variations, the controller 120 may include an output device 130 configured to output data corresponding to a surgery system or surgical procedure, and may comprise one or more of a display device, audio device, and haptic device. The output device 130 may be coupled to a patient platform and/or disposed on a medical cart adjacent to the patient and/or operator. In other variations, the output device 130 may be mounted to any suitable object, such as furniture (e.g., a bed rail), a wall, a ceiling, and may be self-standing.

An output or display device may allow an operator to view images of one or more end effectors, support arms, body cavities, and tissue. For example, an end effector comprising a visualization device (e.g., camera, optical sensor) located in a body cavity or lumen of a patient may be configured to image an internal view of the body cavity or lumen and/or intracavity devices. An external visualization device may be configured to image an external view of the patient and one or more external magnetic positioning devices. Accordingly, the display device may output one or both of internal and external images of the patient and system components. In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

In some variations, the output device 130 may be configured to provide an AR or VR experience for an operator by generating one or more images having enhanced and/or virtual features. These features may comprise graphical overlays onto an image, reference markers associated with objects being imaged (e.g., surgical tools, end effectors, operator hands, etc.), and/or the like to conveniently provide information to an operator during a procedure. A mixed reality experience provided by the output device 130 may help to guide a procedure by providing easily-interpretable, real-time feedback to the operator.

In some variations, the output device 130 may be a combined input/output device, such as a display configured to receive user input and output, for operator interpretation, information related to a patient, surgical procedure, and/or components of the robotic surgery system. For example, the user interface may be configured to provide (e.g., graphically) an indication of one or more dimensions of an end effector that were determined by the processor 124. In some variations, the user interface may be configured to provide one or more (e.g., a series of) prompts for an operator to determine one or more parameters for the surgical procedure.

In some variations, the combined input/output device may be configured to receive a first image (e.g., a real time image captured by an end effector) and simultaneously capture a second image (e.g., an image of a real time field of view of an operator detected by a sensor). The input/output device may be configured to provide the first and second images in combination (e.g., oriented adjacent to each other or overlayed with each other) via a display thereof so that an operator may access both images throughout a surgical procedure. In some variations, the input/output device may comprise an AR or VR tool, such as one or more of an AR or VR headset, goggles, glasses, and contact lens. The AR or VR tool may be configured to provide, via a display, one or more augmented or virtual images received and/or detected by the tool. An augmented image may comprise, for example, a reference marker overlayed onto or adjacent to a desired system component (e.g., a portion of an end effector or surgical instrument) such that the system component may be tracked within the image via the reference marker. In some variations, the reference marker may be generated using an RFID tag located on the end effector or surgical instrument. In some variations, a reference marker may be overlayed onto one or more hands of an operator. The reference marker may comprise one or more of a symbol, image, or text. In some variations, the reference marker may comprise a digital reproduction or digital outline of an end effector or surgical instrument.

In some variations, the output device 130 may comprise audio device configured to audibly output patient data, sensor data, system data, alarms and/or warnings. For example, the audio device may output an audible warning when monitored patient data (e.g., blood pressure) falls outside a predetermined range or when a malfunction in a support arm is detected. As another example, audio may be output when operator input is overridden by the surgery system to prevent potential harm to the patient and/or surgery system (e.g., collision of support arms with each other, excessive force of the intracavity device against a patient cavity wall). In some variations, an audio device may comprise at least one of a speaker, piezoelectric audio device, magnetostrictive speaker, and/or digital speaker. In some variations, an operator may communicate to other users using the audio device and a communication channel. For example, the operator may form an audio communication channel (e.g., VoIP call) with a remote operator and/or observer.

A haptic device may be incorporated into one or more of the input and output devices herein to provide additional sensory output (e.g., force feedback) to the operator. For example, a haptic device may generate a tactile response (e.g., vibration) to confirm operator input to an input device (e.g., touch surface). Haptic feedback may in some variations simulate a resistance encountered by an intracavity device within a body cavity or lumen (e.g., magnetic field and tissue resistance). Additionally, or alternatively, haptic feedback may notify that an operator input is overridden by the surgery system to prevent potential harm to the patient and/or system (e.g., collision of support arms with each other). Operator interaction with a user interface utilizing an input and output device is discussed in more detail herein.

### Methods

The following methods may be used independently or in any combination to prepare for and/or perform robotic surgery. The methods herein may be executed via one or more devices and components of robotic surgery systems described above. In general, a single operator may operate a surgery system or device to execute the methods herein without requiring assistance from another operator to operate the surgery system. In some variations, a single hand, foot, gaze, or tool employed by the operator may be used to perform one or more steps of the methods herein. However, in some variations, the methods may be carried out by two or more operators. While particular steps of the exemplary methods may be described in a particular order, it should be understood that, in some variations, one or more of the steps may rearranged within the method, may be repeated any suitable number of times, or may be optional. Further, in some variations, the methods may include feedback loops and/or additional steps.

FIG. 7 is a flowchart representation of one variation of a method 700 for removably coupling a robotic surgery system. Generally, the method 700 may include removably coupling a robotic surgery system by coupling an end effector connector to a coupling mechanism of a support arm, actuating the coupling mechanism with a first hand of an operator to release the end effector connector from the support arm, and receiving the end effector connector with the first hand of the operator. The method 700 may thus enable single-handed operator end effector exchange and robotic arm control for robotic surgery procedures. However, it should be understood that the method 700 need not be performed by a single hand of the operator. For example, an operator may use a first hand to perform one or more steps of the method 700, and a second hand to perform the remaining steps of the method 700. Additionally, a second operator (or a plurality of secondary operators) may aid a first operator in performing any one of the steps of the method 700.

Moreover, it should be understood that one or more steps of the method 700 may be repeated (e.g., step 706 may be repeated any number of times until a desired actuation is achieved), omitted (e.g., step 702 may be omitted if creating a sterile environment is not desired or required), or reordered. Moreover, while method 700 indicates a feedback loop from step 708 to step 704, it should be understood that this feedback loop is not required (e.g., the feedback loop may be omitted if it is not desired to re-couple the end effector connector and the coupling mechanism), and that additional or alternative feedback loops may be employed to carry out the method 700. Further, one or more steps of method 700 may be performed simultaneously (e.g., step 706 and step 708 may be performed substantially simultaneously, as actuating the actuator may cause the end effector connector to immediately release onto the hand of the operator).

In one variation, method 700 may be used to assemble and disassembly a robotic surgery system. One or more of the steps of method 700 may be performed by a single operator without the aid of another person. In some variations, one or more of the steps may be performed using a single hand (e.g., a first hand) of the single operator. Optionally, method 700 may first include disposing 702 a sterile drape over a support arm. In some variations, the disposing 702 may include disposing he drape over the support arm and a coupling mechanism. The coupling mechanism may be coupled to a distal end of the support arm and may be configured to couple an end effector connector to the support arm. In some variations, the disposing 702 may include orienting the sterile drape between the support arm/coupling mechanism and the end effector connector (e.g., directly between the annular portion of the end effector connector and the sleeve of the coupling mechanism) such that a first (e.g., exterior) side of the drape faces one or more of the end effector connector, end effector, patient, and surgical space, and such that a second (e.g., interior) side of the drape faces the coupling mechanism and the support arm.

Next, method 700 may include coupling 704 the end effector connector to the coupling mechanism. The end effector connector may include: the annular portion and the arm; the annular portion, the arm, and the end effector housing (e.g., via the housing attachment mechanism); or the annular portion, the arm, the end effector housing, and an end effector (e.g., a visualization device or a grasping device). Thus, an optional step for the method 700 may include, either prior to or following the coupling 704, coupling and/or decoupling the end effector or the end effector housing to the end effector connector arm via the housing release/attachment mechanism. In some variations, the coupling 704 may include pressing the annular portion onto and about a circumference of the cylindrical housing. For example, an operator may use a hand (e.g., a first hand) to apply a force to annular portion relative to the coupling mechanism such that the annular portion is translated proximally about the cylindrical portion. The coupling mechanism may include one or more of a cylindrical housing having an actuator disposed therein, a sleeve disposed around the housing, and one or more projections extendable form and retractable into a sidewall of the cylindrical housing. In some variations, the coupling 704 may include coupling the annular portion of the end effector connector around a circumference of the cylindrical housing of the coupling mechanism. In some variations, the coupling 704 may include aligning the annular portion in a particular rotational configuration relative to the sleeve. For example, the particular rotational configuration may be achieved by aligning one or more protrusions and indentations of the annular portion relative to one or more complementary indentations and protrusions of the sleeve. Alternatively, in some variations, the coupling 704 may simply include providing the coupled end effector connector and coupling mechanism. For example, the robotic surgery system may be initially provided as a releasably coupled system, and not as a system of discrete components.

The method 700 may then include actuating 706 the coupling mechanism with a first hand of the operator. For example, the actuating 706 may include actuating an actuator of the coupling mechanism to release the end effector connector from the coupling mechanism. In some variations, the actuating 706 may include applying a force to the actuator with the first hand such that the actuator translates proximally through the cylindrical housing of the coupling mechanism. The applied force may be a continuous force requiring at least a portion of the first hand of the operator to correspondingly advance into the cylindrical housing. As such, the portion of the first hand may become surrounded by the cylindrical housing and the annular portion of the end effector connector coupled thereabout during the actuation 706. That is, the portion of the first hand and the annular portion may be laterally aligned, with the first hand being within an aperture of the annular portion. In alternate variations, the actuating 706 may include using a first tool or suitable replacement for a hand of the operator (e.g., a receptacle having a rigid or semi-rigid projection configured to apply a force to the actuator).

Finally, the method 700 may include receiving 708 the end effector connector with the first hand of the operator. In some variations, the receiving 708 may include catching, grasping, or otherwise collecting the annular portion of the end effector connector with the first hand of the operator. For example, if the actuating 706 includes advancing a portion of the first hand of the operator through the cylindrical housing such that the annular portion at least partially surrounds the first hand, then the annular portion may be ejected directly onto the first hand of the operator. Still, if the actuating 706 does not include a at least partially surrounding the first hand of the operator with the annular portion, the receiving 708 may still be achieved by the first hand of the operator being positioned directly under the annular portion. Thus, an optional step of the method 700 may include positioning the first hand of the operator such that the end effector connector is released directly onto the first hand. Further, following the receiving 708, another optional step for the method 700 may include coupling and/or decoupling the end effector or the end effector housing to the end effector connector arm via the housing release/attachment mechanism.

In some variations, the method 700 may optionally include indicating (e.g., visually, audibly, haptically, etc.) or generating a notification representative of a coupling status of the end effector and support arm. The indicator or notification may be generated in real time and may be interpretable to an operator of the robotic surgery system. For example, a visual indicator such as an LED light may be used to indicate that the end effector connector is coupled to the support arm via the coupling mechanism.

FIG. 8 is a flowchart representation of one variation of a method 800 for using a support arm input device. Generally, the method 800 may include independently receiving one or more support arm control signals via actuation of one or more corresponding actuators using a foot of an operator. The method 800 may thus enable single-footed operator robotic arm movement control to facilitate end effector control during robotic surgical procedures. However, it should be understood that the method 800 need not be performed by a single foot of the operator. For example, an operator may use a first foot to perform one or more steps of the method 800, and a second foot or suitable tool to perform the remaining steps of the method 800. Additionally, a second operator (or a plurality of secondary operators) may aid a first operator in performing any one of the steps of the method 800.

Moreover, it should be understood that one or more steps of the method 800 may be repeated (e.g., step 802 may be repeated any number of times until a desired movement of the support arm is achieved) or omitted. Moreover, while method 800 indicates a feedback loop from step 804 to step 802, it should be understood that this feedback loop is not required (e.g., the feedback loop may be omitted if it is not desired to reposition the support arm). Further, the steps of the method 800 may be performed simultaneously (e.g., step 702 and step 704 may be performed substantially simultaneously, as receiving the one or more support arm control signals may coincide with controlling the movement of the support arm).

In one variation, method 800 may be used with a support arm input device. The support arm input device may generally include a base having a proximal end, a distal end, a lateral portion, and a midfoot portion between the proximal end and the distal end, where the midfoot portion may be configured to receive a midfoot of an operator. In some variations, the support arm input device may include one or more of: a first forefoot actuator coupled to the distal end of the base, a second forefoot actuator coupled to the lateral portion of the base, and a hindfoot actuator coupled to the proximal end of the base. Actuation of the first forefoot actuator may control one or more of pitch, yaw, and roll of the support arm. Actuation of the second support arm signal may control distal translation of the support arm relative to the operator. Actuation of the third support arm signal may control proximal translation of the support arm relative to the operator. In some variations, the input device may additionally include a first forefoot housing and a third forefoot actuator coupled to the first forefoot housing. Actuation of the third forefoot actuator may switch input device control of support arm motion from a first support arm to a second support arm.

First, the method 800 may include receiving 802 a support arm control signal from the support arm input device. In some variations, the control signal may include one or more control signals. For example, the support arm control signal may include one or more of: a first support arm control signal generated by actuation of the first forefoot actuator, a second support arm signal generated by actuation of the second forefoot actuator, and a third support arm signal generated by actuation of the hindfoot actuator, and a fourth support arm control signal generated by actuation of the third forefoot actuator. In some variations, the input device may be configured to control one or more support arms. In some variations, the receiving 802 may include independently receiving one or more support arm control signals. That is, in some variations, only one support arm control signal may be received by the support arm (e.g., via a controller or processor) at a time. In some variations, the receiving 802 may include generating the one or more support arm control signals with at least a portion of a foot (e.g., a first foot) of an operator. In some variations, the receiving 802 may include generating the first support arm control signal by manipulating a forefoot receptacle with a forefoot of the operator.

Next, the method 800 may include controlling 804 a movement of a support arm based on the received support arm control signal. For example, the controlling 804 may include transmitting (e.g., via an RF communication link) a signal indicative of an adjustment or maintenance of a support arm movement to a component (e.g., a motor, a joint) of the support arm. In some variations, the controlling 804 may include processing (e.g., via a processor) the one or more support arm control signals. In some variations, the one or more support arm control signals may be processed independently.

In some variations, the method 800 may optionally include indicating (e.g., visually, audibly, haptically, etc.) or generating a notification representative of a controlled movement of the support arm based on the one or more support arm control signals. The indicator or notification may be generated in real time, and may be interpretable to an operator of the support arm input device. For example, a visual indicator such as an LED light may be used to indicate that the support arm is currently moving. As another example, multiple unique visual indicators (e.g., different colored LED lights) may be employed to indicate the type of controlled movement of the support arm (e.g., rotational vs. translational and/or proximal vs. distal, etc.). Thus, an operator may receive feedback that the support arm is being properly controlled according to their foot motions.

Further, FIG. 18 depicts a flow diagram of a method 1800 for controlling movement of an end effector. The end effector may comprise one or more of a visualization device, a grasper, a retractor, a magnetic positioning device, a sensor, an intracavity device, a delivery device, a retrieval device, a stapler, a clip applier, and an electrocautery hook. In some variations, the visualization device may comprise an endoscope. First, the method 1800 may include providing 1802 an image of a view (i.e., perspective or field of view) of an operator. The image may include an augmented end effector, such as a reproduction of an end effector including a virtual overlay (e.g., a reference marker or tag) on or adjacent to the end effector. For example, the providing 1802 may comprise tracking the end effector, and overlaying a reference marker onto or adjacent to the end effector in real-time. In some variations, the reference marker may comprise one or more of a symbol, image, or text. In some variations, the reference marker may comprise a digital copy and/or digital outline of the end effector. In some variations, tracking the end effector may comprise position (e.g., real-time position) of the end effector when the end effector is both inside of and external to a body of a patient within the view of the operator. The end effector may comprise an RFID tag that enables tracking of the end effector.

The image may be provided via a display, such as a display of an output device of a controller or control system. In some variations, the output device may comprise an input/output control device configured to receive input from the operator that yields movement of the end effector. In some variations, the input/output device may comprise an augmented reality (AR) or virtual reality (VR) tool. Such a tool may be configured to be worn over an eye of the operator. The input/output device may be configured to detect the view of the operator (e.g., via one or more sensors) to capture and generate the image provided in step 1802. In some variations, the input/output device may be a first controller that may be communicably coupled to a second controller via a remote network. The second controller may be configured to receive input from (e.g., a foot of) the operator to activate the first controller. That is, the second controller may be configured to activate or deactivate the first controller. In some variations, the second controller may be a foot switch or pedal.

Next, the method 1800 may include measuring 1804 a gaze of the operator. This may be achieved using the input/output control device. For example, the device may include one or more sensors for monitoring the gaze of the operator. Next, the method 1800 may include actuating 1806 a support arm to move the end effector (coupled to the support arm) based on the measured gaze of the operator. Moving the end effector may comprise moving the end effector from a first (origin) position to a second (destination) position (e.g., at least one second position relative to the first) based on the measured gaze. For example, in some variations, actuating the support arm may comprise directing the gaze of the operator at a virtual actuator that is provided on the image (e.g., via the display of the input/output device) for a time period. The time period, which may be adjustable or predetermined, may be between about 1 second and about 10 seconds. As another example, actuating the support arm may, in some variations, comprise directing the gaze of the operator toward a region of the image, and moving the end effector to a position within a surgical space that corresponds to the region of the image. Similarly, the end effector may be moved via actuation of the support arm (via a support arm control signal) after the gaze is detected for at least a threshold duration (e.g., at least 1 s, at least 2 s, at least 5 s, at least 8 s, at least 10 s, etc.).

As shown in FIG. 18, the method 1800 may be repeated any number of times to continuously update the augmented image (e.g., provide a real-time image) and to move or adjust a position of the end effector within a surgical space (e.g., within a body cavity of a patient). Moreover, in some variations, the method 1800 may include providing, within the image, a plurality of virtual actuators each configured to respond to the gaze of the operator to generate one of a plurality of support arm control signals. In some variations, a first virtual actuator may be configured to translate the support arm along a first axis within the image, and/or a second virtual actuator configured to translate the support arm along a second, different axis within the image. The first virtual actuator may be configured to enable translation of the support arm such that the end effector moves in a first direction along the first axis, and the second virtual actuator may be configured to enable translation of the support arm such that the end effector moves in the first direction along the second axis. Further, the plurality of virtual actuators may further comprise a third virtual actuator configured to enable translation of the support arm such that the end effector moves in a second, opposite direction along the first axis, and/or a fourth virtual actuator configured to enable translation of the support arm such that the end effector moves in the second direction along the second axis. Additionally, or alternatively, the plurality of virtual actuators may comprise at least one fifth virtual actuator configured to enable rotation of the support arm in one or more of pitch, roll, and yaw. For example, the plurality of virtual actuators may comprise any combination (including all of) fifth, sixth, and seventh virtual actuators configured to enable rotation of the support arm in one pitch, roll, and yaw, respectively.

Turning to the method 1900 of FIG. 19, another method for controlling movement of an end effector will be described. First, the method 1900 may include generating 1902 an image comprising one or more different views of a surgical space. The image may be provided via a display of an input/output device as described with reference to method 1800 (e.g., an AR tool). In some variations, a first view of the image may be based on an operator view of the surgical space (e.g., an external view of the patient body and surrounding instruments and environment), and a second view of the image may be based on an end effector (e.g., visualization device) view of the surgical space (e.g., an internal view of the patient body). In some variations, the image may combine at least two of a plurality of views provided within the image. For example, the image may provide a first view that is overlayed onto a second view, or vice versa. In some variations, the image may include one or more virtual actuators for switching between and reoriented views provided in the image. Then, the method 1900 may include identifying 1904 a position of the end effector within one or more of views making up the image. This step may comprise overlaying a reference marker (e.g., symbol, image, and/or text) onto or adjacent to at least a portion of the end effector. In some variations, the reference marker may comprise a digital reproduction or outline of the end effector. In some variations, the reference marker of the end effector may be visible within the image regardless of the end effector's position relative to the body cavity of a patient. Next, the method 1900 may include actuating 1906 a support arm coupled to the end effector to move (adjust a position of) the end effector based on its identified position within one or more of the views provided in the image (e.g., one or both of the first and second views of the image). When the actuating is based on (at least) a view comprising an internal body cavity of the patient, the method 1900 may further comprise estimating the position of at least a portion of the end effector within the body cavity. Additionally, or alternatively, when the actuating is based on (at least) a view of the image comprising an external environment of the patient (e.g., surgical space) of the image, the method may further comprise estimating depth of at least a portion of the end effector within the body cavity.

Like the method 1800, the method 1900 may be repeated any number of times to continuously update the image (e.g., combined image of multiple views) and to move or adjust a position of the end effector within a surgical space (e.g., within a body cavity of a patient). Furthermore, additional steps for either method 1800 or 1900 may include, for example, identifying or tracking a position of one or more hands of the operator within the image. This may be accomplished by overlaying a reference marker, as described above, onto each of the one or more hands of the operator.

Exemplary methods for determining surgical parameters prior to or during a surgical procedure will now be described.

FIG. 20 shows a method 2000 for determining a parameter for robotic surgery. The parameter may include a position of a reference point (e.g., an incision on a patient body) and/or a dimension of a surgical tool, such as an unknown length of an end effector. The method 2000 may first include defining 2002 a first reference point between an end effector and a support arm coupled thereto. The first reference point may comprise a junction between the end effector (e.g., a proximal portion or end thereof) and a support arm (e.g., via a connector). The end effector may comprise one or more of a visualization device, a grasper, a retractor, a magnetic positioning device, a sensor, an intracavity device, a delivery device, a retrieval device, a stapler, a clip applier, and an electrocautery hook. In general, the end effector may have an elongate body with a longitudinal axis. In some variations, the end effector may have a length of about 20 cm and about 60 cm. The length of the end effector may be known or unknown to execute the method 2000. Next, the method 2000 may include positioning 2004 the end effector relative to a surface of a body within a surgical space. The positioning 2004 may be achieved by manually moving, or transmitting an electrical control signal to, a support arm coupled to the end effector. Positioning the end effector may comprise intersecting the surface of the body with a distal tip of the end effector. The body may comprise: a body of a patient (e.g., an incision thereon), an object (e.g., within a surgical space), and/or a marker. In some variations, the object may comprise a planar surface (e.g., a surgical table).

Next, the method 2000 may include acquiring 2006 data indicating a position of the end effector. The position may be relative to the surface of the body. The acquiring 2006 may occur via a processor and memory. In some variations, the processor may initiate the step 2006 in response to a control signal from an operator. For example, an actuator may be configured to receive operator input to cause the processor to acquire the position data. In some variations, the actuator may be a control button on a support arm. In some variations, an indicator of the actuator, such as an LED, may flash or change colors to indicate that a measurement will be or is being taken. Accordingly, an operator may receive feedback from the system that (at least a portion of) a process for determining a surgical parameter is being executed by the system (e.g., by a processor thereof). It should be noted that using the actuator to acquire position data for the support arm and/or end effector, as described above, may be applicable to any of the related methods herein. For example, any one of: defining or determining a first and/or second reference point of an end effector, and defining or determining a position of the support arm and/or end effector within the surgical space, may result from an operator actuating such an actuator.

Finally, the method 2000 may include determining 2008 a second reference point between the end effector and the surface based on the acquired data and the first reference point (e.g., the surgical parameter). This step is described in further detail below with respect to method 2100. In some variations, the second reference point may be used to determine an unknown length of the end effector (e.g., another surgical parameter), as is described with respect to method 2200 below.

In some variations, the positioning 2104 may comprise moving the end effector relative to the body to form the angle. Moving the end effector may comprise intersecting a surface of the body with a distal tip of the end effector. The body may comprise: a body of a patient (e.g., an incision thereon), an object (e.g., within a surgical space), and/or a marker. In some variations, the object may comprise a planar surface (e.g., a surgical table). In some variations, the object may comprise a surface of a support arm. In some variations, moving the end effector may comprise contacting a marker with a distal tip of the end effector. The marker may be a fiducial marker on a body or object surface. In some variations, the method 2100 may include forming an incision on a body of a patient prior to forming the angle relative to the body.

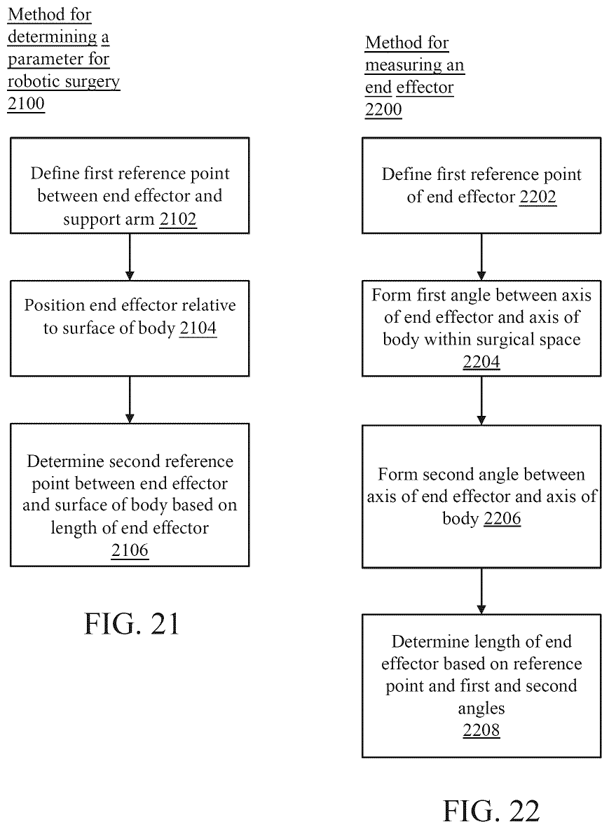

FIG. 21 depicts another method 2100 for determining a parameter for robotic surgery. The method 2100 may be a variation of the method 2000. Like the method 2000, the method 2100 may include defining 2102 a first reference point between an end effector and a support arm coupled thereto and positioning 2104 the end effector relative to a surface of a body within a surgical space. Next, the method 2100 may include determining 2106 a second reference point between the end effector and the surface based on a length of the end effector and the first reference point. The method 2100 may include receiving or determining (e.g., manually measuring) a length of the end effector prior to the determining 2106. The distance between the first reference point and the second reference point may be defined by the length of the end effector, and the position of the second reference point may be defined by this distance. In some variations, the position may be further defined by an angle formed between the end effector and the surface of the body. The position of the second reference point may be determined by triangulating the length of the end effector and a vector defined by the angle between the longitudinal axis of the end effector and the axis of the body. Moreover, surface may be an irregular surface, such as a surface of a patient body (e.g., an incision thereon), or may be planar (e.g., a surface of a surgical table or wall within the surgical space). Further, the end effector may comprise one or more of a visualization device, a grasper, a retractor, a magnetic positioning device, a sensor, an intracavity device, a delivery device, a retrieval device, a stapler, a clip applier, and an electrocautery hook. In general, the end effector may have an elongate body with a longitudinal axis

Turning to FIG. 22, the method 2200 may be used to measure an end effector. The method 2200 may also be a variation of the method 2000. First, the method 2200 may comprise defining 2202 a position of a first reference point of the end effector, which may be a junction between the end effector (e.g., a proximal portion or end thereof) and a support arm (e.g., via a connector). The end effector may comprise one or more of a visualization device, a grasper, a retractor, a magnetic positioning device, a sensor, an intracavity device, a delivery device, a retrieval device, a stapler, a clip applier, and an electrocautery hook. In general, the end effector may have an elongate body with a longitudinal axis. In some variations, the end effector may have a length of about 20 cm and about 60 cm. Next, the method 2200 may include forming 2204 a first angle between a longitudinal axis of the end effector and an axis of a body within a surgical space. The axis may be parallel to a surface of the body. Next, the method 2200 may include forming 2206 a second, different angle between the longitudinal axis of the end effector and the axis of the body. The forming steps 2204, 2206 may comprise moving the end effector to a first or second, different position relative to the body to form first and second angles, respectively. Moving the end effector may comprise intersecting a surface of the body with a distal tip of the end effector. The body may comprise: a body of a patient (e.g., an incision thereon), an object (e.g., within a surgical space), and/or a marker. In some variations, the object may comprise a planar surface (e.g., a surgical table). In some variations, the object may comprise a surface of a support arm. In some variations, moving the end effector may comprise contacting a marker with a distal tip of the end effector. The marker may be a fiducial marker on a body or object surface. In some variations, the method 2200 may include forming an incision on a body of a patient prior to forming the angle relative to the body.

Finally, the method 2200 may include determining 2208 the length of the end effector based on the first reference point and the first and second angles. In some variations, determining the length of the end effector may include determining a second reference point between a distal tip of the end effector and a surface of the body and calculating a distance between the first and second reference points. The second reference point may be determined by triangulating first and second vectors defined by the first and second angles.

In some variations, the step of forming different vectors to solve for unknown variables within the surgical space, as in the methods 2000, 2100 and 2200 above, may be repeated any number of times. As a result, a corresponding number of vectors may be averaged and used to determine the surgical reference point and, in some variations, the length of the end effector, via triangulation. In some variations, the surgical reference point may comprise a point on a second support arm that is adjacent to a first support arm. That is, the methods herein may be used to define a relative position of (a point on) the second support arm and thus the distance between the two support arms. Such a method may help to maintain a desired distance between the support arms during surgery, thereby preventing intra-support arm (or support arm cart) collisions.

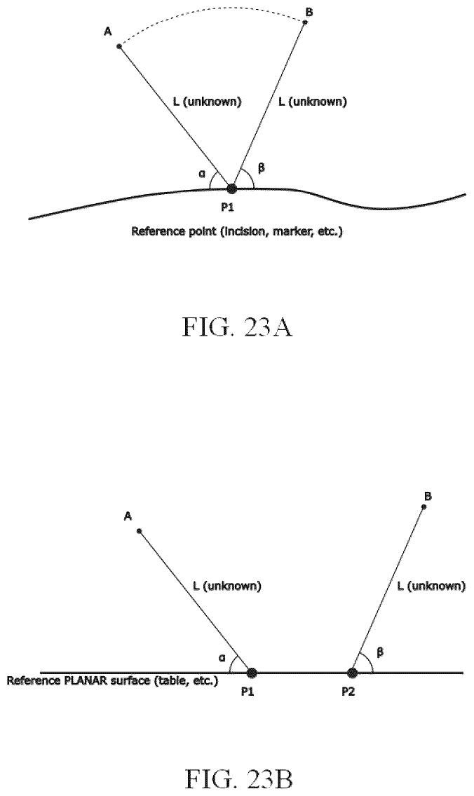

Referring first to FIG. 23A, points A and B may be first reference points defined by an intersection of an end effector with a support arm (e.g., via a connector) and the position of the support arm in space (e.g. a surgical space). Point PI may be a second reference point on a surface of a body. The surface may be irregularly shaped, such as on a body of a patient (e.g., an incision thereof). The line L may correspond to a length of a body of an end effector. The position of the second reference point PI may be defined by, in at least two steps, aligning the end effector with the reference point, determining an angle (alpha, beta, etc.) between the longitudinal axis of the end effector and the surface of the body, and defining a vector based on the determined angle and a corresponding one of the first references points A, B. The aligning may comprise contacting the surface with a distal tip of the end effector. Further, the aligning may comprise repositioning the support arm to change its position (and thus the position of the end effector) relative to the second reference point P1. The angles alpha and beta may be different angles. The at least two vectors may be averaged to determine the position of the second reference point P1, which may be a position that is closest to all defined vectors on average. Next, the length of the line L may be determined by calculating the distance between one of the first reference points A, B and the position of the second reference point P1.

Turning now to FIG. 23B, points A and B may again be first reference points defined by an intersection of an end effector with a support arm (e.g., via a connector) and the position of the support arm in space (e.g. a surgical space). Point P1 may be a second reference point on a surface of a body, and point P2 may be a third reference point on the same surface. The surface may be planar, such as on a surgical table or other planar surface within a surgical space. Accordingly, the points P1 and P2 need not be in the same position (though in some variations, may be in the same position). The line L may correspond to a length of a body of an end effector. The position of the second and third reference points P1, P2 may be defined by, for each point, aligning the end effector with the second or third reference point, determining an angle (alpha or beta) between the longitudinal axis of the end effector and the surface of the body, and defining a vector based on the determined angle and the first reference point. Accordingly, the length of the line L may be determined by calculating the distance between each of the first reference points A, B and the corresponding second or third reference point P1, P2, and then averaging these distances.

Although the foregoing variations have, for the purposes of clarity and understanding, been described in some detail by illustration and example, it will be apparent that certain changes and modifications may be practiced, and are intended to fall within the scope of the appended claims. Additionally, it should be understood that the components and characteristics of the systems and devices described herein may be used in any combination. The description of certain elements or characteristics with respect to a specific figure are not intended to be limiting or nor should they be interpreted to suggest that the element cannot be used in combination with any of the other described elements. For all of the variations described herein, the steps of the methods may not be performed sequentially. Some steps are optional such that every step of the methods may not be performed.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

Additionally, it should be appreciated that ranges disclosed herein may be exemplary, and include all ranges and subranges therein.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1.-22. (canceled)

23. A support arm input device, comprising:
a base comprising a proximal end, a distal end, a lateral portion, and a midfoot portion between the proximal end and the distal end, the midfoot portion configured to receive a midfoot of an operator;
a first forefoot actuator coupled to the distal end of the base, the first forefoot actuator configured to generate a first support arm control signal corresponding to one or more of pitch, yaw, and roll of a support arm;
a second forefoot actuator coupled to the lateral portion of the base, the second forefoot actuator configured to generate a second support arm control signal corresponding to distal translation of the support arm relative to the operator; and
a hindfoot actuator coupled to the proximal end of the base, the hindfoot actuator configured to generate a third support arm control signal corresponding to proximal translation of the support arm relative to the operator.

24. The device of claim 23, wherein the first forefoot actuator comprises a first forefoot housing releasably coupled to a first forefoot receptacle configured to receive the forefoot of the operator, the first forefoot housing comprising a plurality of forefoot switches.

25. The device of claim 24, wherein each switch of the plurality of forefoot switches is configured to be actuated via manipulation of the first forefoot receptacle by the forefoot of the operator.

26. The device of claim 23, wherein the first forefoot actuator is configured to control one or more of pitch, yaw, and roll movement of an end effector of the support arm.

27. The device of claim 23, wherein the second forefoot actuator comprises a second forefoot switch configured to be actuated by a forefoot of an operator.

28. The device of claim 23, wherein the second forefoot actuator comprises a second forefoot housing configured to receive the forefoot of the operator.

29. The device of claim 23, wherein the second forefoot actuator is configured to control distal translation of an end effector of the support arm relative to the operator.

30. The device of claim 23, wherein the second forefoot actuator is coupled to a distal end of the lateral portion.

31. The device of claim 23, wherein the hindfoot actuator comprises a hindfoot switch configured to be actuated by a hindfoot of the operator.

32. The device of claim 31, wherein the hindfoot switch comprises an adjustment mechanism configured to adjust a position the hindfoot switch along a longitudinal axis of the base.

33. The device of claim 23, wherein the hindfoot actuator comprises a hindfoot housing configured to receive a hindfoot of the operator.

34. The device of claim 23, wherein the hindfoot actuator is configured to control proximal translation of an end effector of the support arm relative to the operator.

35. The device of claim 23, wherein each of the first forefoot actuator, the second forefoot actuator, and the hindfoot forefoot actuator is configured to be independently actuated.

36. The device of claim 23, wherein the support arm comprises a first support arm, wherein the device is configured to control movement of the first support arm and a second support arm, and wherein the device further comprises:
a third forefoot actuator coupled to a first forefoot housing, wherein the third forefoot actuator is configured to generate a fourth support arm control signal for transferring transmissions of the first, second, and third support arm control signals from the first support arm to a second support arm.

37. The device of claim 36, wherein the third forefoot actuator comprises a third forefoot switch configured to be actuated by an underfoot of the operator.

38. The device of claim 36, wherein the third forefoot actuator is coupled to an exterior surface of a first forefoot housing of the first forefoot actuator.

39. The device of claim 23 in a robotic surgery system, the system further comprising:
the support arm; and
an end effector releasably couplable to the support arm.

40. The device of claim 39, wherein the end effector comprises one or more of a visualization device, a grasper, a retractor, a magnetic positioning device, a sensor, an intracavity device, a delivery device, a retrieval device, a stapler, a clip applier, and an electrocautery hook.

41. The device of claim 39 in the robotic surgery system, the system further comprising an end effector connector configured to releasably couple the end effector to the support arm.

42. The device of claim 39, wherein the support arm control signal is configured to control movement of one or both of the support arm and the end effector.

43. A method for using a support arm input device comprising a base having a proximal end, a distal end, a lateral portion, and a midfoot portion between the proximal end and the distal end, the midfoot portion configured to receive a midfoot of an operator, method comprising:
independently receiving one or more of:
a first support arm control signal via actuation of a first forefoot actuator coupled to the distal end of the base;
a second support arm control signal via actuation of a second forefoot actuator coupled to the lateral portion of the base; and
a third support arm control signal via actuation of a hindfoot actuator coupled to the proximal end of the base; and
controlling a movement of a support arm relative to a surgical space based on one or more of the first, second, and third support arm control signals,
wherein the first support arm signal controls one or more of pitch, yaw, and roll of the support arm,
wherein the second support arm signal controls distal translation of the support arm, and
wherein the third support arm signal controls proximal translation of the support arm.

44.-131. (canceled)
